SEQUENCE LISTING (17 sequences)

SEQ ID NO: 1 - RNA, 238 nt, synthetic construct
  source 1..238: mol_type = other RNA
ggccatgtgtatgtggggagacggtcgggtccagatattcgtatctgtcgagtagagtgtgggctccccacatactttgttgatccggatcccgactggcgagagccaggtaacgaatggatccggatcaatcatggccgggaaatgcatgtctaagacagcatcttcgcgtgttatggttgtgaaatggtacctgtgaagccacagatggggtgttatggttgtgaaatggtactgc

SEQ ID NO: 2 - RNA, 262 nt, synthetic construct
  source 1..262: mol_type = other RNA
ggccatgtgtatgtggggagacggtcgggtccagatattcgtatctgtcgagtagagtgtgggctccccacatactttgttgatccgcggggacgaggacgagcacttcttctgtgaagccacagatgggaagaagtgctcgtcctcgtccctgcggatcaatcatggccgggaaatgcatgtctaagacagcatcttcgcgcaaagtgtgtaacggaatactgtgaagccacagatgggtattccgttacacactttgtgc

SEQ ID NO: 3 - RNA, 262 nt, synthetic construct
  source 1..262: mol_type = other RNA
ggccatgtgtatgtggggagacggtcgggtccagatattcgtatctgtcgagtagagtgtgggctccccacatactttgttgatccgcgcaaagtgtgtaacggaatactgtgaagccacagatgggtattccgttacacactttgtgcggatcaatcatggccgggaaatgcatgtctaagacagcatcttcgcgaactgtacaaactactacctcactgtgaagccacagatgggtgaggtagtagtttgtacagtttgc

SEQ ID NO: 4 - DNA, 402 bp, Qubevirus durum
  source 1..402: mol_type = genomic DNA
atggcaaaattagagactgttactttaggtaacatcgggaaagatggaaaacaaactctggtcctcaatccgcgtggggtaaatcccactaacggcgttgcctcgctttcacaagcgggtgcagttcctgcgctggagaagcgtgttaccgtttcggtatctcagccttctcgcaatcgtaagaactacaaggtccaggttaagatccagaacccgaccgcttgcactgcaaacggttcttgtgacccatccgttactcgccaggcatatgctgacgtgaccttttcgttcacgcagtatagtaccgatgaggaacgagcttttgttcgtacagagcttgctgctctgctcgctagtcctctgctgatcgatgctattgatcagctgaacccagcgtattga

SEQ ID NO: 5 - DNA, 708 bp, synthetic construct
  source 1..708: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 6 - RNA, 22 nt, unidentified
  source 1..22: mol_type = other RNA
gtaccatttcacaaccataaca

SEQ ID NO: 7 - RNA, 19 nt, Homo sapiens
  source 1..19: mol_type = other RNA
tattccgttacacactttg

SEQ ID NO: 8 - RNA, 22 nt, synthetic construct
  source 1..22: mol_type = other RNA
aagaagtgctcgtcctcgtccc

SEQ ID NO: 9 - RNA, 22 nt, Homo sapiens
  source 1..22: mol_type = other RNA
tgaggtagtagtttgtacagtt

SEQ ID NO: 10 - RNA, 38 nt, synthetic construct
  source 1..38: mol_type = other RNA
ggatcccgactggcgagagccaggtaacgaatggatcc

SEQ ID NO: 11 - RNA, 49 nt, synthetic construct
  source 1..49: mol_type = other RNA
gagacggtcgggtccagatattcgtatctgtcgagtagagtgtgggctc

SEQ ID NO: 12 - protein, 13 aa, synthetic construct
KYGRRRQRRKKRG

SEQ ID NO: 13 - DNA, 31 bp, synthetic construct
  source 1..31: mol_type = other DNA
gtgggaattccatggcaaaattagagactgt

SEQ ID NO: 14 - DNA, 23 bp, synthetic construct
  source 1..23: mol_type = other DNA
caccaagctttcaatacgctggg

SEQ ID NO: 15 - DNA, 34 bp, synthetic construct
  source 1..34: mol_type = other DNA
gtggagatcttaatacgactcactatagggccat

SEQ ID NO: 16 - DNA, 30 bp, synthetic construct
  source 1..30: mol_type = other DNA
caccctcgagcaaaaaacccctcaagaccc

SEQ ID NO: 17 - DNA, 24 bp, synthetic construct
  source 1..24: mol_type = other DNA
gcacaaagtgtgtaacggaatacc

(19) United States
(12) Patent Application Publication
(10) Pub. No.: US 2025/0332279 A1
(43) Pub. Date: Oct. 30, 2025

(54) PHARMACEUTICAL COMPOSITION AND METHOD FOR TREATING GLIOBLASTOMAS

(71) Applicant: National Cheng Kung University, Tainan City (TW)

(72) Inventors: Hong-Wei YANG, Tainan City (TW); Hao-Han PANG, Tainan City (TW); Ying-Pei HSU, Tainan City (TW); Nan-Si LI, Tainan City (TW)

(73) Assignee: National Cheng Kung University, Tainan City (TW)

(21) Appl. No.: 19/189,112

(22) Filed: Apr. 24, 2025

Related U.S. Application Data
(60) Provisional application No. 63/638,627, filed on Apr. 25, 2024.

(51) Int. Cl. A61K 47/69; A61K 31/7105; A61K 47/64; A61P 35/00; C12N 7/00; C12N 15/113; C12N 15/115

(52) U.S. Cl. CPC A61K 47/6901; A61K 31/7105; A61K 47/645; A61P 35/00; C12N 7/00; C12N 15/1138; C12N 15/115; C12N 2310/14; C12N 2310/16; C12N 2310/52; C12N 2310/531; C12N 2795/18023

(57) ABSTRACT

The present invention provides a bioengineered bacteriophage-like nanoparticle, rQβ@b-3WJ, comprising a Qβ capsid and a 3WJ RNA scaffold (b-3WJ), which is a three-way junction motif integrated with (1) a Qβ phage capsid binding hairpin; (2) a light-up aptamer; and (3) a structural siRNA element (siRNA₁); wherein the Qβ phage capsid binding hairpin is further integrated with a different structural siRNA element (siRNA₂). Also provided is a nanoparticle, TrQβ@b-3WJ, comprising a Qβ capsid conjugated with TAT peptides and a 3WJ RNA scaffold (b-3WJ), which enhances cellular internalization for highly efficient gene silencing.

### CROSS REFERENCE TO RELATED APPLICATION

This non-provisional application claims the benefit under 35 U.S.C. § 119(e) on U.S. Patent Provisional Application No. 63/638,627 filed on Apr. 25, 2024, the entire contents of which are hereby incorporated by reference.

### FIELD OF THE INVENTION

This invention provides a new construct for treating Glioblastomas (GBMs).

### REFERENCE TO ELECTRONIC SEQUENCE LISTING

The application contains a Sequence Listing which has been submitted electronically in .XML format and is hereby incorporated by reference in its entirety. Said .XML copy, created on Apr. 23, 2025, is named “YHW0001US Sequence Listing.xml” and is 17,892 bytes in size. The sequence listing contained in this .XML file is part of the specification and is hereby incorporated by reference herein in its entirety.

### BACKGROUND OF THE INVENTION

Glioblastomas (GBMs) are primary brain tumors characterized by aggressive growth and rapid recurrence. Newly diagnosed GBM patients who receive debulking surgery and adjuvant radiotherapy combined with Temozolomide (TMZ) chemotherapy, both treatments inducing DNA damage, have an average survival of 14.6 months. Despite aggressive treatment, patients always succumb to recurrence-related death, especially radiation resistance. Radiation resistance in GBMs is generally attributed to the hypoxic tumor microenvironment, which creates insufficient oxygen supply thereby rendering tumors highly resistant to radiation-induced killing through rapid DNA repair. Previous studies have also suggested that CD133-positive tumor cells represent the cellular population that confers glioma radiation resistance and could be the source of tumor recurrence after radiation. In addition, chemotherapy and radiation therapy-induced stress can lead to dedifferentiation of tumor cells to a glioma stem cells (GSCs)-like state, and the GSCs have been shown to promote tumor recurrence.

GBMs respond to DNA damage induced by ionizing radiation (IR) and TMZ treatment through increased expression of DNA repair enzymes, including the proteins O-6-methylguanine-DNA methyltransferase (MGMT) and poly-(ADP-ribose) polymerase 1 (PARP-1). Thus, there is an urgent need to quickly identify the molecular basis of therapy resistance in the primary tumors of GBM patients and develop strategies to abrogate the repair by effectively knocking down the target genes in primary and/or recurrent tumors. RNA nanotechnology has been growing rapidly as a new generation platform for specific RNA target suppression. As nanotechnology rapidly evolves, encapsulation of small interfering RNA (siRNA) in nanoparticles is a promising way to improve the effectiveness of siRNA for cancer treatment using lipid-, polymer-, metal-, and virus-based nanoparticles. However, the application of nanoparticle-encapsulated siRNA is confined by its low targeting efficiency, chemical and thermodynamic instability, and poor biocompatibility. Currently, three-way junction (3WJ) RNA nanoparticles to deliver microRNA (miRNA)/silencing RNA (siRNA) have been reported to address thermodynamic instability issues. The thermodynamically stable 3WJ motif derived from the bacteriophage phi29 DNA packaging motor (pRNA) core is composed of three oligos with a branched structure. In particular, various sequences of siRNA can easily be incorporated into the branch of the pRNA-3WJ motifs via bottom-up self-assembly, which could be processed intracellularly by Dicer for multigene silencing. In addition, the RNA backbones with 2′-fluorine, 2′-O-methyl or 2′-amine modifications of U and C nucleotides render the RNAs resistant to RNase degradation or hydrolysis, enhancing their in vivo half-life while retaining authentic functions of the incorporated modules. However, the chemical modification of RNAs will affect the folding properties and biological functions of RNA molecules and will also cause higher production costs and lower production yields. Moreover, when the structure of the RNA becomes more complex to have more functions, the challenge above will be more critical. Virus-like particles (VLPs) are constructed from viral structural proteins and capsomers and are free of any genetic material. They are genome-free versions of their viral nanoparticle (VNP) counterparts and are considered noninfectious, nontoxic, and nonimmunogenic. Viruses are regarded as naturally occurring nucleic acid carriers, as they protect and carry their cargo. Furthermore, drugs can also be infused, encapsulated, absorbed, or conjugated to the interior and exterior surfaces of coat protein interfaces through attachment to various functional groups offered by the protein structure. This flexibility offers a variety of possibilities, including reversible binding of active molecules, protection within proteinaceous matrices, and specific targeting to the site of action. In addition, VLPs are devoid of their own genome; they can easily encapsulate nucleic acids and therefore have been broadly used for the delivery of genes as well as therapeutic nucleic acids. Tian et al. conjugated the transacting activation transduction (TAT) peptide onto the exterior surface of tobacco mosaic virus (TMV), which exhibited enhanced internalization for miRNA delivery. Lam et al. also used cowpea chlorotic mottle virus (CCMV) VLPs carrying a cell penetrating peptide(M-lycotoxin peptide L17E) to enhance siRNA delivery into mammalian cells.

It is desirable to develop a new approach for treating GBMs.

### SUMMARY OF THE INVENTION

Accordingly, the present invention provides a bioengineered bacteriophage-like nanoparticle as RNAi Therapeutics, which can enhance radiotherapy against cancers or other diseases.

In one aspect, the present invention provides a bioengineered bacteriophage-like nanoparticle, rQβ@b-3WJ, comprising a Qβ capsid and a 3WJ RNA scaffold (b-3WJ), in which the b-3WJ is a three-way junction motif integrated with (1) a Qβ phage capsid binding hairpin; (2) a light-up aptamer; and (3) a first structural siRNA element (siRNA₁); wherein the Qβ phage capsid binding hairpin is further integrated with a second structural siRNA element (siRNA₂).

In one embodiment of the present invention, the rQβ@b-3WJ further comprises a transactivating transcriptional activator (TAT) peptide. One example of the present invention is a bioengineered bacteriophage-like nanoparticle comprising a Qβ capsid conjugated with a TAT peptide and a 3WJ RNA scaffold (b-3WJ), which is a three-way junction motif integrated with (1) a Qβ phage capsid binding hairpin; (2) a light-up aptamer; and (3) a first structural siRNA element (siRNA₁); wherein the Qβ phage capsid binding hairpin is further integrated with a second structural siRNA element (siRNA₂).

In one further aspect, the present invention provides a pharmaceutical composition, comprising a therapeutically effective amount of the bioengineered bacteriophage-like nanoparticle according to the present invention, wherein the 3WJ RNA scaffold has an RNA sequence selected from the group consisting of SEQ ID NO:2 and SEQ ID NO:3.

According to the invention, the bioengineered bacteriophage-like nanoparticle nanoparticles have the benefits as follows: (1) biological production of all components and self packaging, (2) multigene silencing, (3) real-time monitoring of intracellular RNA cleavage by Dicer enzyme using fluorescence microscopy, (4) RNA stability enhancement, and (5) great biosafety.

In one embodiment of the invention, the nanoparticles are incorporated with EGFR siRNA and miRNA Let-7g into the 3WJ RNA scaffold to produce rQβ@b-3WJsiEGFR+Let-7g, which can be delivered to tumor tissues through the convection-enhanced delivery (CED) method to overcome the blood-brain barrier (BBB) challenge and enhance radiotherapy in GBMs.

In one yet aspect, the present invention provides a new construct containing 3WJ RNA scaffold (b-3WJ) integrated with a nucleic acid bioproduction and self-packaging system to produce the nanoparticles according to the invention, e.g. b-3WJ packaged red-fluorescent Qβ VLPs (also called as hereinafter TrQβ@b-3WJsiEGFR+Let-7g), which considerably enhances antitumor efficacy via the synergistic effect of silencing the multigene related to DNA repair promotion, cell invasion ability, and radiotherapy, providing a promising approach for treating GBMs.

In one embodiment of the invention, the light-up aptamer is malachite green (MG) aptamer or broccoli aptamer.

In one embodiment of the invention, the structural siRNA element comprises EGFR siRNA, LUC2 siRNA, and Let-7g.

In one embodiment of the invention, the sequence of the 3WJ RNA scaffold is of a DNA sequence selected from the group consisting of SEQ ID NO:2 and SEQ ID NO:3.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one drawing executed in color. Copies of this patentor patent application publication with color drawing(s) will be provided by the Office upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred.

In the drawings:

FIGS. 1(a) to 1(e) provide the design and characterization of the Qβ@b-3WJsiSCR+MG. FIG. 1(a) provides the simulation of the secondary structure of the b-3WJsiSCR+MG scaffold. The simulated Gibbs free energy is −130.70 kcal/mol. FIG. 1(b) provides the transmission electron microscopy (TEM) image of WT-Qβ and Qβ@b-3WJsiSCR+MG. Scale bar represents 200 nm. FIG. 1(c) provides the U rea-PA G E electrophoresis analysis of the bioproduced b-3WJsiSCR+MG RNA scaffold. RNA extracted from E. coli was loaded in different lanes. The gel was stained with DFHBI-1T (right) for imaging the 3WJ RNA scaffold and SY BR green II (left) for imaging total RNA. L: ladder. Lane 1: control (E. coli without VLP production plasmid). Lane 2: E. coli without IPTG induction. Lane 3: E. coli with IPTG induction. Lane 4: in vitro transcription product. Red arrow: predicted RNA band (237 nt). Blue arrow: RNA polymerase read-through T7 terminator caused a larger fragment. FIG. 1(d) provides the bright view images, fluorescence images and fluorescence spectra for Qβ@b-3WJsiSCR+MG co-incubated with DFHBI-1T. Ex=418 nm. FIG. 1(e) provides the bright view images, fluorescence images, and fluorescence spectra for Qβ@b-3WJsiSCR+MG co-incubated with malachite green. Ex=560 nm.

FIGS. 2(a)-2(d) provide the quantification of RNA scaffolds in VLPs by single-molecule photobleaching imaging. FIG. 2(a) provides the experimental design to monitor the stepwise fluorescence photobleaching of individual Qβ@b-3WJsiSCR+MG. FIG. 2(b) provides the fluorescence image of immobilized individual Qβ@b-3WJsiSCR+MG obtained under our system. The enlarged image indicates a single Qβ@b-3WJsiSCR+MG marked with a red square. FIG. 2(c) shows the representative fluorescence time trace of (i) one DFHBI-1T/RNA complex with one-step photobleaching (P=76.2%), (ii) two DFHBI-1T/RNA complexes with two step photobleaching (P=12.4%), (iii) one DFHBI-1T/RNA complex without photobleaching within acquisition time window (P=11.4%). FIG. 2(d) provides the Fluorescence intensity histograms of (i) two DFHBI-1T/RNA complexes in oneQβ@b-3WJsiSCR+MG before the first-step photobleaching (6872±1,892), (ii) two DFHBI-1T/RNA complexes in one Qβ@b-3WJsiSCR+MG before the second-step photobleaching (3889±1,000), (iii) one DFHBI-1T/RNA complex in one Qβ@b-3WJsiSCR+MG before the first-step photobleaching (4360±773), (iv) one DFHBI-1T/RNA complex in one Qβ@b-3WJsiSCR+MG without photobleaching within acquisition time window (3838±1,400).

FIGS. 3(a) to 3(g) provide the design and characterization of rQβ@b-3WJsiEGFR+siLUC. FIG. 3(a) shows the simulation of the secondary structure of the b-3WJsiEGFR+siLUC scaffold. The simulated Gibbs free energy is −157.70 kcal/mol. FIG. 3(b) provides the TEM image of rQβ@b-3WJsiEGFR+siLUC. Scale bar represents 200 nm. FIG. 3(c) shows the Urea-PAGE electrophoresis analysis of bioproduced b-3WJsiEGFR+siLUC RNA scaffold. RNAs extracted from Qβ VLPs was loaded in different lanes. The gel was stained with DFHBI-1T (right) for imaging the 3WJ RNA scaffold and SY B R green II (left) for imaging total RNA. L: ladder. Lane 1: in vitro transcription product. Lane 2: rQβ@b-3WJsiEGFR+siLUC. Lane 3: Qβ VLPs. Red arrow: predicted RNA band (260 nt). The band vaguely visible at approximately 300 nt indicates that the T7 terminator caused a larger fragment. FIG. 3(d) provides MALDI-TOF Ms spectrum of Qβ@b-3WJsiEGFR+siLUC. FIG. 3(e) provides MALDI-TOF Ms spectrum of TQβ@b-3WJsiEGFR+siLUC. FIG. 3(f) provides the characterization of prepared rQβ@b-3WJsiEGFR+siLUC (Lane 1) and TQβ@b-3WJsiEGFR+siLUC (Lane 2) by 15% SDS-PAGE. The molecular weight of the QβCP monomer is 14.4 kDa. Blue arrow: QβCP monomer; dark blue arrow: QβCP dimer; yellow arrow: QβCP monomer conjugated with TAT peptide; brown arrow: QβCP dimer conjugated with TAT peptide. FIG. 3(g) shows the three-dimensional U87MG cell spheroid uptake of (i) PBS (control), (ii) rQβ@b-3WJsiEGFR+siLUC, and (iii) TrQβ@b-3WJsiEGFR+siLUC. Blue channel: Hoechst 33342-stained cell nucleus; green channel: DFHBI-1T incubated 3WJ RNA s containing broccoli aptamer; red channel: mCherry protein (rQβ VLPs), merge: the merged image of the three channels above. Scale bar: 200 μm.

FIGS. 4(a) to 4(e) provide the RNA scaffold degradation in vitro. FIG. 4(a-c) shows Northern blotting for total RNA extracted from treated cells. FIG. 4(a) provides SY BR green II-stained 10% Urea-PAGE. Lane L: low range ssRNA ladder, Lane 1: RNA extracted from WT-Qβ (negative control), Lane 2: blank (untreated cells), 3: TrQβ VLPs, 4: TrQβ@b-3WJsiEGFR+siLUC (24 h), Lane 5: TrQβ@b-3WJsiEGFR+siLUC (26 h), Lane 6: TrQβ@b-3WJsiEGFR+siLUC (36 h), Lane 7: TrQβ@b-3WJsiEGFR+siLUC (48 h). FIG. 4(b) provides the Northern blotting for the lanes shown in FIG. 4(a) using Urea-PAGE by biotin-conjugated siEGFR complementary DNA probe. b-inner box is the detailed image of the black box in FIG. 4(b); α to ε indicate different sizes of R N A segments. FIG. 4(c) provides the quantitation of different lanes in FIG. 4(b) using ImageJ. FIG. 4(d) shows the RNase III process assay of relative fluorescence intensity and image comparison of naked 3WJ RNAs before and after the RNase III process. Values are expressed as the means±standard deviations (SDs)(n=3). *Indicates a significant difference (Student's t-test, *p<0.05). FIG. 4(e) provides the real-time image of TrQβ@b-3WJsiEGFR+siLUC-treated cells after DFHBI-1T incubation. Scale bar represents 20 μm. Blue: Hoechst 33342-stained nucleus, red: mCherry protein in TrQβ@b-3WJsiEGFR+siLUC, green: broccoli aptamer after incubation with DFHBI-1T and merged is the merge of all channels with bright view.

FIGS. 5(a) to 5(e) provide the in vitro functional analysis of b-3WJ RNA scaffolds. FIG. 5(a) shows the Luciferase reporter assay and luciferase Western blot analysis of U87MG cells after treatment with WT-Qβ or TrQβ@b-3WJsiEGFR+siLUC. Values are expressed as the means±SDs (n=3). *Indicates a significant difference (Student's t-test, *p<0.05). FIG. 5(b) shows the simulation of the secondary structure of b-3WJ Let-7g siEGFR scaffold. The simulated Gibbs free energy is −156.48 kcal/mol. FIG. 5(c) provides IKKα and NF-κB (p65) protein Western blot analysis of U87MG cells after treatment with PBS (Lane1), 1 μM of TrQβ VLPs (Lane 2) or rQβ@b-3WJsiEGFR+Let-7g (Lane 3) or TrQβ@b-3WJsiEGFR+Let-7g (Lane 4). FIG. 5(d) provides the Immunofluorescence staining images of EGFR expression in U87MG cells treated with TrQβ VLPs, rQβ@b-3WJsiEGFR+siLUC or TrQβ@b-3WJsiEGFR+siLUC for 72 h. Scale bar represents 20 μm. FIG. 5(e) provides the immunofluorescence staining images of NF-κB expression in U87MG cells treated with TrQβ VLPs, rQβ@b-3WJsiEGFR+siLUC or TrQβ@b-3WJsiEGFR+siLUC for 72 h. Scale bar represents 60 μm.

FIGS. 6(a) to 6(f) show the in vivo tumor inhibition and survival studies. FIG. 6(a) shows the distribution of delivered rQβ@b-siEGFR+Let-7g in brain tumor tissue after 0.5, 2, and 24 h via CED infusion. FIG. 6(b) shows the distribution of delivered TrQβ@b-3WJsiEGFR+Let-7g in brain tumor tissue after 0.5, 2, and 24 h via CED infusion. FIG. 6(c) shows the treatment protocols assessing multigene silencing-enhanced radiotherapy. FIG. 6(d) provides the brain magnetic resonance imaging (MRI) images of NU mice after treatments. Control: PBS injection only. TrQβ VLPs: Qβ VLP (5 μM) injection only. Radiotherapy: 2Gy X-ray irradiation only. Carboplatin+2Gy X-ray irradiation: carboplatin (10 mg/mL) injection followed by 2Gy X-ray irradiation. rQβ@b-3WJsiEGFR+Let-7g (5 μM)+2Gy X-ray irradiation: rQβ@b-3WJsiEGFR+Let-7g (5 μM) injection followed by 2Gy X-ray irradiation. TrQβ@b-3WJsiEGFR+Let-7g (5 μM) and 2Gy X-ray irradiation: TrQβ@b-3WJsiEGFR+Let-7g (5 μM) injection followed by 2Gy X-ray irradiation. FIG. 6(e) shows tumor volume measurement by MRI for mice after different treatments. *Indicates a significant difference (Student's t-test, *p<0.05) of TrQβ@b-si EGFR+Let-7g (5 μM) and 2Gy X-ray irradiation to all other treatment groups on Day 41. There is no significant tumor volume difference between all groups on Day 5. FIG. 6(f) shows the Kaplan-Meier survival curves for mice that received different treatments. Values are expressed as the means±SDs (n=10). * indicates a significant difference (log-rank test, *p<0.05) of TrQβ@b-3WJsiEGFR+Let-7g (5 μM) and 2Gy X-ray irradiation to all other treatment groups.

FIG. 7 shows the detailed gene silencing mechanisms of TrQβ@b-3WJ Let-7g+siEGFR toward GBM cells. After cell internalization, TrQβ@b-3WJsiEGFR+Let-7g will be disassembled to release b-3WJsiEGFR+Let-7g. The b-3WJsiEGFR+Let-7g will then be processed by the Dicer enzyme and form the RISC to target both EGFR (by siEGFR) and IKKα (by Let-7g). Downregulation of EGFR causes inhibition of cell growth and migration, and the IKK pathway is one of the downstream pathways of EGFR. The inhibition of IKK formation by Let-7g downregulates the NF-κB DNA damage repair process for gene silencing-enhanced radiotherapy in GBMs.

FIGS. 8(a) and 8(b) provide the designs of the dual-plasmid coexpression production system of the b-3WJ RNAi scaffold containing Qβ VLPs in the invention, FIG. 8(a) provides the design of the dual-plasmid coexpression production system of the b-3WJ RNAi scaffold containing Qβ VLPs in this project for Qβ VLPs without mCherry protein. FIG. 8(a) provides the design of the dual-plasmid coexpression production system of the b-3WJ RNAi scaffold containing Qβ VLPs in this project for Qβ VLPs with mCherry protein expression. The product shown in FIG. 8(a) contains only the b-3WJ RNAi scaffold (Qβ@b-3WJ), while the product shown in FIG. 8(b) contains the scaffold and mCherry inside (rQβ@b-3WJ) for further tracing applications.

FIG. 9 shows the dynamic light scattering (DLS) of WT-Qβ and Qβ@ b-3WJsiEGFR+MG.

FIG. 10 shows the calibration curve of the b-3WJ RNA scaffold with DFHBI-1T coincubation to quantify the number of RNAi scaffolds for each Qβ VLP. Values are expressed as the means±SDs (n=3).

FIGS. 11(a) and 11(b) shows the Fluorescence spectra of VLP-based samples. FIG. 11(a) shows the Fluorescence spectra of VLP-based samples under 560 nm. FIG. 11(b) shows the Fluorescence spectra of VLP-based samples under 418 nm excitation.

FIG. 12(a)-12(b) show the stability studies of b-3WJ RNAs. FIG. 12(a) shows the result of Time-dependent fluorescence (Ex: 418 nm, Em: 510 nm) for 144 h with or without urea (biological concentration 10 mM). Values are expressed as the means±SDs (n=3). FIG. 12(b) shows the result of Time-dependent fluorescence (Ex: 418 nm, Em: 510 nm) intensity of b-3WJ RNAs packaged in Qβ VLPs (black) or naked RNA (red). Values are expressed as the means±SDs (n=3).

FIG. 13 shows the Western blotting of EGFR expression in TrQβ@b-3WJsiEGFR+sLUC-treated U87MG cells with dose-dependent expression inhibition.

FIG. 14 shows the fluorescence spectra and image (Eₓ: 418 nm, Eₘ: 510 nm) of TrQβ@b-3WJsiEGFR+Let-7g after incubation with DFHBI-1T.

FIG. 15 shows the stability of TrQβ@b-3WJsiEGFR+Let-7g in solution with 0.1 mg/mL RNase A (Eₓ: 418 nm, Eₘ: 510 nm).

FIGS. 16(a) to 16(c) show the flow cytometry analysis of EGFR (left chart), IKKα (middle chart), and NFκB (right chart) expression in U87MG cells incubated with 1 μM of WT-Qβ, rQβ@b-3WJsiEGFR+Let-7g, and TrQβ@b-3WJsiEGFR+Let-7g.

FIGS. 17(a) and 17(b) show the wound healing assay for U87MG cells after WT-Qβ, rQβ@b-3WJsiEGFR+siEGFR or TrQβ@b-3WJsiEGFR+Let-7g treatment for 72 h. FIG. 17(a) provides the images showing the results of the wound healing assay for U87MG cells. FIG. 17(b) shows the quantification results. Values are expressed as the means±SDs (n=3). *Indicates a significant difference (Student's t-test, *p<0.05).

FIG. 18 shows the trans-well assay for U87MG cells treated with WT-Qβ, rQβ@b-3WJsiEGFR+Let-7g or TrQβ@b-3WJsiEGFR+Let-7g.

FIG. 19 shows the cell growth assay for U87MG cells treated with WT-Qβ, rQβ@b-3WJsiEGFR+Let-7g or TrQβ@b-3WsiEGFR+J Let-7g for 72 h. Values are expressed as the means±SDs (n=8). *Indicates a significant difference (Student's t-test, *p<0.05).

FIG. 20 shows the investigation of in vitro synergistic effect of EGFR/IKKα gene silencing and X-ray irradiation. Values are expressed as the means±SDs (n=8). *Indicates a significant difference (Student's t-test, *p<0.05).

FIG. 21 shows the endotoxin assay of purified WT-Qβ. The endotoxin in WT-Qβ (5 μM) was analyzed using Lonza LAL endotoxin assay kit by measuring the value of OD_{450 nm} and compared with the endotoxin standard sample (positive control, 50 EU/mL). Values are expressed as the means±SDs (n=3). * indicates a significant difference (Student's t-test, *p<0.05).

FIG. 22 shows the cell viability of U87MG cells treated with different concentrations of WT-Qβ (without 3WJ RNAs) for 24 h by X TT assay (n=8).

FIG. 23 shows the distribution of delivered b-siEGFR+Let-7g after CED infusion into brain tissue for 0.5 h, 1 h, and 2 h. The tissues were then stained with and diamidino-2-phenylindole (DAPI) for nuclear staining.

FIG. 24 shows the Western blotting of NF-κB (P65) expression in tumour tissue cells from U87MG tumour-bearing mice that received rQβ@b-3WJsiEGFR+Let-7g or TrQβ@b-3WJsiEGFR+Let-7g via CED infusion.

FIG. 25 shows the inhibition of NF-κB (P65) by gene silencing therapy using WT-Qβ, rQβ@b-3WJsiEGFR+Let-7g, or TrQβ@b-3WJsiEGFR+Let-7g. Three weeks after CED injection, tumors were sectioned and stained with a mouse anti-human P65 antibody, reflecting NF-κB (P65) downregulation of tumor cells. Representative images show immunohistochemistry staining of the tumor tissues from the mice treated with PBS as control, WT-Qβ, rQβ@b-3WJsiEGFR+Let-7g, and TrQβ@b-3WJsiEGFR+Let-7g.

FIG. 26 shows the tumor volume measurement by MRI for mice after different treatments. Values are expressed as the means±SDs (n=10). *Indicates a significant difference (Student's t-test, *p<0.05).

FIG. 27 shows brain damage and toxicity evaluation using H&E staining from CED treatment with saline, TrQβ VLPs, and TrQβ@b-3WJsiEGFR+MG infusion, as shown in three representative sections of brains after 5 days.

FIG. 28 shows the body weight of mice that received different treatments. Values are expressed as the means±SDs (n=8). *Indicates a significant difference (Student's t-test, *p<0.05).

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereto known to those skilled in the art.

The present invention provides a bioengineered bacteriophage-like nanoparticle, rQβ@b-3WJ, comprising a Qβ capsid and a 3WJ RNA scaffold (b-3WJ), which is a three-way junction motif integrated with (1) a Qβ phage capsid binding hairpin; (2) a light-up aptamer; and (3) a structural siRNA element (siRNA₁); wherein the Qβ phage capsid binding hairpin is further integrated with a different structural siRNA element (siRNA₂).

The present invention also provides a bioengineered bacteriophage-like nanoparticle, TrQβ@b-3WJ, comprising a Qβ capsid conjugated with TAT peptides and a 3WJ RNA scaffold (b-3WJ), which is a three-way junction motif integrated with (1) a Qβ phage capsid binding hairpin; (2) a light-up aptamer; and (3) a structural siRNA element (siRNA₁); wherein the Qβ phage capsid binding hairpin is further integrated with a different structural siRNA element (siRNA₂).

As used herein, the term “structural siRNA element” refers to a short interfering RNA (siRNA) that is integrated into the 3WJ scaffold as a component and maintains a secondary structure contributing to scaffold integrity and gene-silencing function. The structural siRNA element in the present invention including but not limited to EGFR siRNA, LUC2 siRNA, and Let-7g.

As used herein, the term “light-up aptamer” refers to a nucleic acid aptamer that becomes fluorescent upon binding to a specific small-molecule fluorophore. The fluorescence is negligible or absent when the fluorophore is unbound. Exemplary light-up aptamers include, but are not limited to, the malachite green (MG) aptamer, which fluoresces upon binding to malachite green dye, and the Broccoli aptamer, which activates fluorescence in the presence of DFHBI (3,5-difluoro-4-hydroxybenzylidene imidazolinone).

As used herein, the “transactivating transcriptional activator” or “TAT” refers to a transactivating transcriptional activator from human immunodeficiency virus 1 (HIV-1) that could be efficiently taken up from the surrounding media by numerous cell types in culture. The number of known CPPs has expanded considerably, and small molecule synthetic analogues with more effective protein transduction properties have been generated

As used herein, the term “pharmaceutical composition” refers to a composition or a formulation comprising the nanoparticles according to the invention, which can be prepared by conventional methods. For example, the pharmaceutical composition may be prepared by mixing the nanoparticles according to the invention with optional pharmaceutically acceptable carriers, including solvents, dispersion media, isotonic agents and the like. The carrier can be liquid, semi-solid or solid carriers. In some embodiments, carriers may be water, saline solutions or other buffers (such as serum albumin and gelatin), carbohydrates (such as monosaccharides, disaccharides, and other carbohydrates including glucose, sucrose, trehalose, mannose, mannitol, sorbitol, or dextrins), gel, lipids, liposomes, resins, porous matrices, binders, fillers, coatings, stabilizers, preservatives, antioxidants (including ascorbic acid and methionine), chelating agents (such as EDTA), salt forming counter-ions (such as sodium), non-ionic surfactants (such as TWEEN™, PLURONICS™ or polyethylene glycol (PEG)], or combinations thereof.

The term “pharmaceutically acceptable carrier” used herein refers to a carrier(s), diluent(s) or excipient(s) that is acceptable, in the sense of being compatible with the other ingredients of the formulation and not deleterious to the subject to be administered with the pharmaceutical composition. Any carrier, diluent or excipient commonly known or used in the field may be used in the invention, depending to the requirements of the pharmaceutical formulation.

According to the invention, the pharmaceutical composition may be adapted for administration by any appropriate route, including but not limited to systematic, oral, rectal, nasal, topical, vaginal, or parenteral route. Such formulations may be prepared by any method known in the art of pharmacy.

The term “treating” as used herein refers to the application or administration of a composition including one or more active agents to a subject afflicted with a disease, a symptom or conditions of the disease, or a progression of the disease, with the purpose to cure, heal, alleviate, relieve, alter, remedy, ameliorate, improve, or affect the disease, the symptoms or conditions of the disease, the disabilities induced by the disease, or the progression of the disease.

The term “subject” as used herein includes human or non-human animals, such as companion animals (e.g. dogs, cats, etc.), farm animals (e.g. cattle, sheep, pigs, horses, etc.), or experimental animals (e.g. rats, mice, guinea pigs, etc.).

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function.

The term “Qβ phage capsid binding hairpin” as used herein refers to a specific RNA secondary structure motif that is recognized by the Qβ bacteriophage coat protein during capsid self-assembly. This hairpin typically contains a stem-loop structure with a conserved sequence and folding pattern that facilitates high-affinity binding to Qβ coat proteins, enabling selective encapsidation of RNA molecules into Qβ virus-like particles (VLPs).

According to the present invention, the sequences of the 3WJ RNA scaffolds of bioengineered bacteriophage-like nanoparticles are provided as follows:

| Name of nanoparticle | Sequence of 3WJ RNA scaffold |
|---|---|
| Qβ@b-3WJsiSCR + MG | SEQ ID NO: 1 |
| rQβ@b-3WJsiEGFR + siLUC | SEQ ID NO: 2 |
| TrQβ@b-3WJsiEGFR + siLUC | |
| rQβ@b-siEGFR + Let-7g | SEQ ID NO: 3 |
| TrQβ@b-siEGFR + Let-7g | |

According to the invention, a construct containing (1) and (called as “TrQβ@b-siEGFR+Let-7g”) was successfully prepared as a genetic therapeutic, which can efficiently knock down EGFR and IKKα simultaneously and inactivate NF-κB signaling, thereby inhibiting DNA repair in a highly efficient manner for enhancing radiotherapy. It was ascertained that the status of released b-3WJ siEGFR+Let-7g processed into its mature form by Dicer for gene silencing can be easily and real-time monitored using fluorescence microscopy. The TrQβ@b-3WJsiEGFR+Let-7g showed a robust ability to protect packaged RNA scaffolds from unwanted threats (i.e., enzymatic digestion), high tumor cell penetration efficiency, and surrounded the whole tumor with a high concentration of b-3WJsiEGFR+Let-7g by CED infusion, which can bypass the blood-brain barrier BBB to reduce systemic toxicity. Conspicuously, these genetic therapeutics provide a chance to serve as a powerful gene-silencing enhanced radiotherapy for clinical GBM treatment and other brain diseases of the central nervous system.

The present invention is further illustrated by the following examples, which are provided for the purpose of demonstration rather than limitation.

### Example

### Plasmid Construction for Qβ VLPs and 3WJ RNA Scaffolds.

The Qβ Coat protein (QβCP) expression vector pCDF-QβCP was constructed according to our previous study. The QβCP DNA sequence (SEQ ID NO:4) and mCherry red fluorescent protein DNA sequence (SEQ ID NO:5) were inserted into the pCDFDuet-1 plasmid. The mCherry DNA sequence was originally from plasmid pmCherry. After cloning into the pET28-b(+) plasmid using BamHI and NotI restriction enzymes for insertion, the mCherry sequence was cloned into pCDFDuet-1 to produce the plasmid pCDFDuet-1-mCherry. The QβCP sequence was originally from pCDFDuet-1-QβCP-GFP, which has been described previously and was inserted into the pCDFDuet-1-mCherry plasmid using EcoNI and the XhoI restriction site to produce the protein expression vector pCDFDuet-1-QβCP-mCherry. The multifunctional 3WJ RNA scaffold gene sequence was purchased from GENEWIZ and cloned into the pET28-b(+) plasmid using the BglII and XhoI restriction sites to produce the RNA scaffold expression vector pET28-b(+)-3WJ. The primers used in this study are listed in the table below.

TABLE 1

| Primer ID | Sequence (5′-3′) | SEQ ID NO |
|---|---|---|
| Qβ_FWD | GTGGGAATTCCATGGCAAAATTA | SEQ ID NO: 13 |
| | GAGACTGT | |
| Qβ_REV | CACCAAGCTTTCAATACGCTGGG | SEQ ID NO: 14 |
| T7_FWD | GTGGAGATCTTAATACGACTCAC | SEQ ID NO: 15 |
| | TATAGGGCCAT | |
| T7_REV | CACCCTCGAGCAAAAAACCCCTC | SEQ ID NO: 16 |
| | AAGACCC | |

### Bioproduction of 3WJ Scaffold Packaging VLPs.

The vectors pCDF-QβCP (stpR) and pET28-b(+)-3WJ (KanR) were transformed into E. coli strain BL21 (H IT-21, RBC, USA) to produce 3WJ scaffolds packaged in VLPs (Qβ@b-3WJ) without fluorescent protein. For coexpression of QβCP, mCherry protein and the 3WJ RNA scaffold, pCDFDuet-1-QβCP-mCherry (stpR) and pET28-b(+)-3WJ (KanR) were transformed into E. coli BL21 cells (HIT-21, RBC, USA) for expression. E. coli BL21 cells harboring the plasmids were grown in either LB broth supplemented with antibiotic (streptomycin) at 50 μg/mL. Starter cultures were grown for 18 h at 37° C. and were used to inoculate 1 L of expression culture. IPTG (1 nM) was used as a protein expression reagent at an OD600 nm of 0.8-1.0 in LB broth. The IPTG-supplemented culture was incubated at 37° C. overnight for approximately 16-18 h. The overnight culture was harvested by centrifugation at 6500 g, resuspended in 20 mL of PBS buffer (pH=7.4) and then lysed by sonication. The lysate was centrifuged for 30 min at 23000 g, followed by ammonium sulfate precipitation, which was used to obtain crude VLPs. Crude VLPs were resuspended in PBS buffer, followed by 20% w/v PEG8000-NaCl precipitation to obtain pure VLPs. VLPs were resuspended in 1 mL PBS buffer and extracted with 1:1 n-butanol:chloroform. The VLP-based samples from the aqueous layer were purified by step sucrose gradient ultracentrifugation and then precipitated with 20% w/v PEG8000/2 M NaCl solution and resuspended in 25 mL of PBS buffer, followed by exhaustive dialysis (SnakeSkin Dialysis Tubing, 10,000 MWCO. Thermo, LOT: QD213952, USA) against PBS buffer (pH=7.4) for 48 h. The obtained pure VLP-based samples were concentrated using protein concentrate filter tubes (Amicon Ultra15 Centrifugal Filter Units. 100,000 MWCO. Merck Millipore, LOT: R6EA45140, Ireland). The final concentration of VLPs was assessed using a Pierce BCA Protein Assay kit (Thermo, LOT: PD202250, USA).

### TAT-Peptide Conjugation Procedure and Analysis.

The functional peptide cys-TAT was conjugated on the surface of rQβ@b-3WJ to enhance cell uptake. The Cys-TAT peptides (KYGRRRQRRKKRG(SEQ ID NO:12)-cys-SH) were conjugated to rQβ@b-3WJ by sulfosuccinimidyl 4-(N-maleimidomethyl) cyclohexane-1 carboxylate (sulfo-SM CC; Sigma-Aldrich, St. Louis, MO, USA) as a cross-linker. Briefly, 5 μL of sulfo-SM CC solution (10 mg/mL in deionized (DI)-H2O) was added to a 600 μL solution of 2 μM rQβ@b-3WJ in PBS buffer (pH=7.4) for 30 min at 25° C. in the dark and then purified using a filter column (Amicon Ultra15 Centrifugal Filter Units. 100,000 MWCO. Merck Millipore, LOT: R6EA45140, Ireland) with PBS buffer. The samples were desalted with a filter column (100,000 MWCO) and washed 3 times with PBS buffer. Subsequently, the maleimide-terminated rQβ@b-3WJ was reacted with 30 μL of Cys-TAT solution (0.3 mg/mL) at 25° C. for 2 h in the dark and then purified again using the above-mentioned procedure to obtain TrQβ@b-3WJ.

To confirm the successful conjugation of TAT peptides on rQβ@b-3WJsiEGFR+siLUC. The rQβ@b-3WJsiEGFR+siLUC and TrQβ@b-3WJsiEGFR+siLUC were mixed with 2-mercaptoethanol (SigmaAldrich, St. Louis, MO, USA) and incubated at 95° C. for disulfide bond breaking and protein denaturing. The denatured samples were analyzed by SDS-PAGE (12%) electrophoresis and then stained using Coomassie Brilliant Blue R-250 Dye(Sigma-Aldrich, St. Louis, MO, USA).

Dynamic Light Scattering Characterization. The diameters of VLP-based samples in PBS buffer (pH=7.4) were analyzed by dynamic light scattering (DLS). Two hundred microliters of the VLP based sample (Qβ VLPs and Qβ@b-3WJsiSCR+MG) solution was added to a 3-open microvolume cuvette for diameter analysis.

### Transmission Electron Microscopy (TEM).

Five microliters of VLP-based samples were pipetted onto Formvar-coated copper mesh grids (400 mesh, Ted Pella, Redding, CA, USA) for 5 min, followed by exposure to 8 μL of a solution of uranyl acetate (15 mg/mL in D I H2O) for 2 min as a negative stain. Excess stain was then removed, and the grids could dry in air for 10 min.

### In-Gel Urea Page Electrophoresis of RNA Scaffolds.

In vitro transcribed b-3WJ RNA scaffolds were prepared following the protocol of the HiScribe T7 High Yield RNA Synthesis Kit (N EB, USA). The packaged b-3WJ RNA scaffolds were prepared by extracting the b-3WJ RNA scaffolds from Qβ@b-3WJ according to our previously described methods. Purified RNAs (1 μg/well) were electrophoresed with 8% urea page at 90 V for 4 h. After washing with DI-H2O, the gel was stained with DFHBI-1T to observe the broccoli aptamer (SEQ ID NO:11), followed by SY BR green II staining to observe the total RNA.

### RNA Scaffold Fluorescence Assay.

Purified Qβ@b-3WJ was resuspended in RNA aptamer binding buffer (40 mM 4-(2-hydroxyethyl)-1-piperazineethanesulfonic acid) HEPES, 100 mM KCl, 1 mM MgCl2, pH=7.4), coincubated with 10 μM DFHBI-1T for 30 min at 37° C. and subjected to UV-vis spectrometry to measure the maximum absorbance wavelength as the fluorescence excitation wavelength. Fluorescence intensity measurement was performed using an M2 enzyme-linked immunosorbent assay (ELISA) spectrometer (Molecular Device, Silicon Valley, CA, USA).

Stability Studies of the b-3WJ RNA Scaffold Packaged in Qβ VLPs.

In vitro transcribed b-3WJ RNA scaffolds were produced by the method mentioned previously using the HiScribe T7 High Yield RNA Synthesis Kit (N EB, USA). Approximately 1 μM naked b-3WJ RNA or 1 μM packaged b-3WJ RNA was pretreated with 10 μM DFHBI-1T in RNA binding buffer and incubated at 37° C. for 30 min followed by mixing with various concentrations of Rnase A. The fluorescence intensity (Ex=418 nm, Em=510 nm) was then analyzed using an M2 ELISA spectrometer to estimate the stability of naked b-3WJ RNA and packaged b-3WJ RNA at different time points. We also investigated the stability of naked b-3WJ RNA and packaged b-3WJ RNA after treatment with 10 mg/mL urea for 0, 1, 2, 4, 15, 24, 48, 72, 96, 120, and 144 h. Then, the fluorescence intensity was analyzed to estimate the stability of naked b-3WJ RNA and packaged b-3WJ RNA at different time points by an M2 ELISA spectrometer.

### In Vitro Cell Culture.

The glioma cell Line U87MG was cultured in Dulbecco's modified Eagle's medium (DMEM) supplemented with 2.2 mg/mL sodium carbonate, 10% fetal bovine serum (FBS), 50pg/mL gentamicin, 50 μg/mL penicillin, and 50pg/mL streptomycin. Cells were harvested by 0.05% trypsin-ethyldiaminetetraacetic acid (EDTA) solution and washed with PBS buffer (pH=7.4) three times before seeding into experimental wells. Cell Uptake of VLP-Based Samples by U87MG Tumor Spheroids. U87MG cells were cultured at 5×104 cells per well in U end 96-well plates for 72 h to form a spheroid 3D culture. One micromolar VLP-based samples (rQβ@b-3WJsiEGFR+siLUC or TrQβ@b-3WJsiEGFR+siLUC) were added to the cells and incubated for another 24 h. The cell nuclei were stained with Hoechst 33342, and the b-3WJ RNA scaffolds were stained with DFHBI-1T followed by PBS wash. The distribution of Qβ VLPs and delivered b-3WJsiEGFR+siLUC were monitored using laser scanning confocal microscopy. Broccoli Aptamer Tracking Images in Live Cells. U87MG cells were seeded in a glass-based chamber (˜1×105 cells per well) and incubated for 24 h. TrQβ@b-3WJsiEGFR+siLUC (1 μM) was then added and incubated for another 24 h. The medium was removed, and the cells were washed with PBS followed by incubation with DFHBI-1T containing medium. The cells were imaged using 3D-Cell Explorer-Fluo microscopy (Nanolive) at 60×, and images were taken every 10 min for cell nuclei (blue color), Qβ VLPs (red color), and b-3WJ RNA scaffolds (green color). The images were merged and analyzed using STEVE Microscopy software (NanoLive).

### Northern Blot Analysis.

U87MG cells were seeded in 12-well plates (˜1×105 cells per well) and incubated for 24 h. The TrQβ VLPs (1 μM) or TrQβ@b-3WJsiEGFR+siLUC (1 μM) were then added to the culture medium. The cells were harvested after 24, 26, 36, and 48 h of incubation, and the small RNAs were extracted with the mirVana PARIS kit (Life Technologies, Carlsbad, CA, USA), resolved by denaturing gel electrophoresis (urea PAGE), transferred to a Hybond-N+ membrane (G E Healthcare) by the capillary method and immobilized by UV transillumination (320 nm). Northern blotting was performed according to the manufacturer's protocols (North2− South Chemiluminescent Hybridization and Detection Kit, Thermo Scientific, USA). The membrane was probed with a biotin-labeled DNA oligonucleotide (5′-GCA CAA AGT GTG TAA CGG AAT ACC (SEQ ID NO:17) [Biotin]-3′, high performance liquid chromatography (HPLC) purified, Mission Bio, Inc. Taiwan), which is complementary to the EGFR siRNA. The blotting images were analyzed using ImageJ software to quantify the different length fragments of the RNAs.

### Western Blot Analysis.

For in vitro Western blotting, U87MG cells (6×10⁴ per well) in 6-well plates treated with 1 μM VLP-based samples (TrQβ VLPs, TrQβ@b-3WJsiEGFR+siLUC, rQβ@b-3WJsiEGFR+siLUC, and TrQβ@b-3WJsiEGFR+Let-7g) were harvested and washed with PBS (pH=7.4). The cells were treated with PRO-PREP Protein Extraction Solution (iNtRON) to extract proteins, and the protein concentration was quantified using a Pierce bicinchoninic acid (BCA) Protein Assay Kit (Thermo). Proteins were electrophoresced using an 8% SDS-PAGE gel (approximately 20 μg per lane) and transferred to a polyvinylidene fluoride (PVDF) membrane. After blocking with blocking solution (5% milk, 0.1% Tween-20 in TBS buffer, pH=7.4), the beta-actin internal control was stained with beta-actin monoclonal antibody (CAT: 66009-1-Ig, Proteintech, 1/10000 dilution), glyceraldehyde-3-phosphate dehydrogenase (GA PDH) was stained with antiGA PDH antibody (clone 2D9. CAT: TA802519, Invitrogen, 1/5000 dilution). Target protein EGFR was stained with EGFR antibody [GT133](CAT: GTX 628887, GeneTex, 1/2500 dilution), NF-κB (P65) was stained with NF-κB p65/ReIA antibody (CAT: A19653, Abclonal, 1/1000 dilution), and luciferase was stained with antifirefly luciferase antibody [Luci17](ab16466, Abcam, 1/1000 dilution). A goat anti-mouse IgG (H+L)-HRP antibody was used as the secondary antibody for all proteins mentioned. Chemiluminescence signals were imaged using a ChemiDoc™ XRS imaging system. For in vivo Western blotting, the brain tumor tissues of mice treated with 5 μM rQβ@b-3WJsiEGFR+Let-7g or TrQβ@b-3WJsiEGFR+Let-7g were cut into small pieces and carefully washed in 3 mL PBS, then homogenized on ice by a Polytron blender in lysis buffer supplemented with a protease inhibitor cocktail. The homogenates were centrifuged at 2000 rpm for 10 min. at 4° C., and the supernatant was assayed for total protein concentration by BCA Protein Assay Kit and stored at −80° C. until NF-κB analysis using Western blotting was performed as described above.

### Luciferase Reporter Assay.

Luciferase-stable U87MG cells treated with 1 μM VLP-based samples (WT-Qβ and TrQβ@b-3WJsiEGFR+siLUC) for 72 h were harvested and washed with PBS (pH=7.4). The luciferase expression analysis process of luciferase-stable U87MG cells generally followed the Luciferase RGA high sensitivity, 200 assays (Roche) protocol. The chemiluminescence was detected by an M2ELISA spectrometer.

### Flow Cytometry Analysis.

U87MG cells (6×104 per well) in 6-well plates were treated with 1 μM VLP-based samples (TrQβ VLPs, rQβ@b-3WJsiEGFR+Let-7g, and TrQβ@b-3WJsiEGFR+Let-7g) for 72 h were washed three times with 2% FBS contained PBS (pH=7.4). After that, all samples were fixed by 4% paraformaldehyde for 15 min and permeabilized by 0.1% triton X-100 solution for 15 min, the cells were separated into three groups (105 cells/mL for each). Then, those cells were blocked by 1% BSA solution for 20 min, followed by incubation with EGFR antibody (1/1000 dilution, EGFR Rabbit Ab, CAT: A11351, ABClonal), IKKα antibody (1/1000 dilution, IKKα Rabbit mAb, CAT: A19694, ABClonal), or NF-κB antibody (1/1000 dilution, NF-κB p65/ReIA Rabbit mAb, CAT: A19653, ABClonal) for 1.5 h. The cells were further incubated with secondary antibody (1/500 dilution, 488-conjugated Goat Anti-Rabbit IgG (H+L), CAT: AS053, ABClonal) for 1 h. All samples were quantified by Atune Nxt flow cytometer (Thermo Fisher Scientific, USA).

### Immunofluorescence Microscopy.

U87MG cells (3×104 per well in a 24-well plate) were treated with 1 μM VLP-based samples (TrQβ VLPs, rQβ@b-3WJsiEGFR+Let-7g, and TrQβ@b-3WJsiEGFR+Let-7g) for 72 h. Then, the cells were washed with PBS (pH=7.4) and fixed with 75% ethanol. Then, the cells were incubated in blocking solution (10% bovine serum albumin (BSA), 0.3 M glycine and 0.1% Tween-20 in PBS buffer, pH=7.4) for 1 h. The blocked cells were incubated with EGFR antibody (ab8465, 1/1000 dilution) overnight at 4° C. The cells were further incubated with goat antimouse IgG (H+L)-FITC secondary antibody (1/1000 dilution). The nuclei were stained using Hoechst 33342, and images were taken using fluorescence microscopy.

### In Vitro Cell Studies.

The cultured U87MG cells (5,000 cells/well) were treated with Qβ VLPs with final concentrations of 0.5, 1.0, 2.0, and 4.0 μM followed by incubation for 24 h to verify the cytotoxicity of Qβ VLPs. The culture medium was removed, and the cells were incubated in 120 μL of XTT solution for 2 h. After that, 100 μL of XTT solution from each well was transferred to another 96-well counting plate. The survival of U87MG cells was evaluated by GD at 490 nm using a SpectraMax M2 microtiter plate reader. U87MG cells were treated with 1 μM of VLP-based samples (WTQβ, rQβ@b-3WJsiEGFR+Let-7g, and TrQβ@b-3WJsiEGFR+Let-7g) for 72 h and then seeded into 96-well plates (1000 cells per well). After 72 h of incubation, the cells were washed with PBS buffer (pH=7.4) and incubated with XTT solution at 37° C. for 30 min (n=8). Cell growth rate was analyzed by measuring the absorbance at 450 nm using an SpectraMax M2 microtiter plate reader. To investigate the in vitro synergistic effect of EGFR/IKKα gene silencing and X-ray irradiation, U87MG cells (5,000 cells per well) were treated with 1 μM of TrQβ@b-3WJsiEGFR+Let-7g for 72 h followed by 2Gy X-ray irradiation. After 24 h, the cells were washed with PBS buffer (pH=7.4) and incubated with XTT solution at 37° C. for 30 min (n=8). Cell viability was analyzed by measuring the absorbance at 450 nm using an SpectraMax M2 microtiter plate reader. Cell viability (%) was defined as the relative absorbance of the treated samples versus that of the untreated controls.

### Transwell Migration Assay.

Transwell assays generally followed the Cell Migration, Chemotaxis and Invasion Assay using the Staining protocol (Corning, NY, USA). U87MG cells were treated with VLP based samples (TrQβ VLPs, rQβ@b-3WJsiEGFR+Let-7g, and TrQβ@b-3WJsiEGFR+Let-7g) for 72 h. Then, the treated cells were harvested, resuspended in F B S-free D M E M, and seeded at 2×104 cells in the inset of a 24-well Transwell plate. The reservoir volume was 0.65 mL of DM EM containing 10% FBS in each well. After 4 h of incubation, the interior of the Transwell membrane was wiped, and the migrated cells were stained with Giemsa stain and imaged with microscopy.

### Endotoxin Assay.

Briefly, 100 μL of 2-Mercaptoethanol (20 mM) was mixed with 100 μL of Qβ VLPs (10 μM) or 100 μL of endotoxin standard (100 EU/mL; Endotoxin, E. coli 055:B5, cat: 193783) at 37° C. for 30 min followed by filtration using A micon Ultra-0.5 Centrifugal Filter Unit (UFC5003, Millipore, USA). Then the endotoxin concentration in the elutriant (from Qβ VLPs or endotoxin standard) was then analyzed by Kinetic-QC L Kinetic Chromogenic LAL Assay Kit.

### Animal Procedures.

For the animal experiments, luciferase expression plasmid-transfected U87MG cell-implanted pathogen-free male NU/NU mice (5-7 weeks old, 20-25 g, from BioLASCO, Taiwan) were employed in this study. U87MG cells were cultured at 37° C. with 5% CO₂ in M EM with 10% fetal bovine serum and 1% penicillin/streptomycin (Invitrogen). Mice were anaesthetized with 2% isoflurane gas and immobilized on a stereotactic frame to implant U87MG cells. A sagittal incision in the skin overlying the calvarium was created. U87MG cell implantation was performed by creating a hole in the exposed cranium 1.5 mm anterior and 2 mm lateral to the bregma using a 27G needle. A total volume of 5 μL of U87MG cell suspension (1×10⁵ cell/μL) was injected at a depth of 3 mm from the brain surface over a 5 min period. The needle was withdrawn over 2 min. MRI was performed to monitor brain tumor growth for 7 days after tumor cell implantation.

### CED Procedures.

The details of the CE D procedure are as described in our previous study. Briefly, infusion cannulas were fabricated with silica tubing (Polymicro Technologies, Phoenix, AZ, USA) fused to a 0.1 mL syringe (Plastic One, Roanoke, VA, USA) with a 0.5 mm stepped-tip needle that protruded from the silica guide base. The treatment agents (VLPs or drugs) were loaded into the syringes and attached to a microinfusion pump (Bioanalytical Systems, Lafayette, IN, USA). The syringe with a silica cannula was mounted onto a stereotactic holder and then lowered through a puncture hole made in the skull to the implanted tumor. The sample solution was infused at a rate of 1 μL/min until a volume of 5 μL had been delivered, and the cannula was removed 2 min later.

### In Vivo Antitumor Efficiency of Gene Silencing-Enhanced Radiotherapy.

All animal experiments were approved by the Animal Committee of Chang Gung University and adhered to the experimental animal care guidelines (IACUC NO. CGU106-036). Mice were raised in a room with a thermostat at 26° C. Nu/Nu mice weighing approximately 25-30 g (5-6 weeks old) were tested to confirm the efficacy of the proposed approach. Intracranial brain tumors were induced by transplantation of U87MG cells into mouse brains. Briefly, cultured U87MG cells (5×105 cells/mouse) were injected over a 2 min period into the brain using a syringe, and needle withdrawal was conducted over another period of 0.5 min. A total of 50 mice were used, and the experiments were divided into five groups. In Group 1 (n=10), the mice received no further treatment after transplantation of U87MG cells, and these mice served as the control. In Group 2 (n=10), the mice received TrQβ VLPs (5 μM) via CED infusion two times (every 7 days) after 7 days of U87MG cell transplantation. In Group 3 (n=10), the mice received X-ray irradiation (2Gy) two times (every 7 days) after 7 days of U87MG cell transplantation. In Group 4 (n=10), the mice received carboplatin (10 mg/mL) via CED infusion at 9 A.M. followed by 2Gy X-ray irradiation at 2 P.M. the following day. Seven days after U87MG cell transplantation, the treatment was repeated two times (every 7 days). In Group 5 (n=10), the mice received TrQβ@b-3WJsiEGFR+Let-7g (5 μM) via CED infusion at 9 A.M. Seven days after U87MG cell transplantation, the treatment was repeated two times (every 7 days). In Group 6 (n=10), the mice received rQβ@b-3WJsiEGFR+Let-7g (5 μM) via CED infusion at 9 A.M. followed by 2Gy X-ray irradiation at 2 P.M. the following day. Seven days after U87MG cell transplantation, the treatment was repeated two times (every 7 days). In Group 7 (n=10), the mice received TrQβ@b-3WJsiEGFR+Let-7g (5 μM) via CED infusion at 9 A.M. followed by 2Gy X-ray irradiation at 2 P.M. the following day. Seven days after U87MG cell transplantation, the treatment was repeated two times (every 7 days). The intracranial tumor volume in mice was measured by MR imaging. The survival time was calculated from the day of U87MG cell inoculation (0 day) to the day of death. Kaplan-Meier survival curves were plotted for each group. The body weight of the mice was monitored at determined time intervals.

### Histopathological Studies.

Histopathological studies were performed on 10-μm sections of paraformaldehyde-fixed, paraffin embedded mouse brains. Slides were soaked in hydrochloric acid-potassium ferrocyanide solution for 30 min at room temperature. The distribution of delivered b-3WJsiEGFR+Let-7g conjugated with DFHBI-1T (green fluorescence) was monitored through fluorescence microscopy imaging after staining nuclei with DAPI. The images were taken after 0.5, 1, and 2 h of CED infusion. For brain tissue damaging situation evaluation, brain tissues from the mice after 5 days of different treatments [5 μL of saline, 5 μL of TrQβ VLPs (5 μM), and 5 μL of TrQβ@b-3WJsiSCR+MG (5 μM)]were stained by hematoxylin and eosin (H& E).

### Statistical Analysis.

The data were expressed as the mean±SD on the basis of at least three independent experiments. Statistical analysis was performed using Student's t-test and log-rank test. Differences were considered statistically significant if p<0.05.

### Results

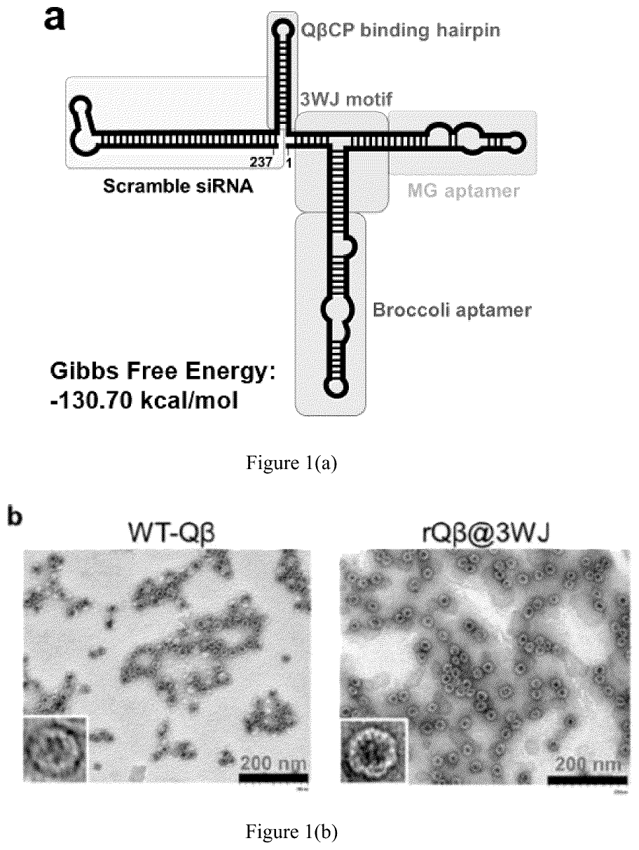

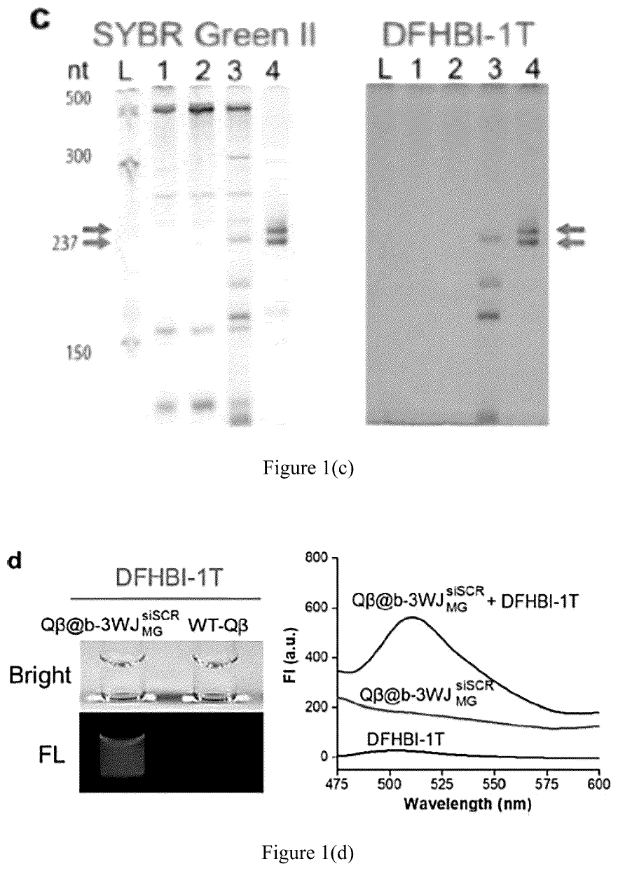

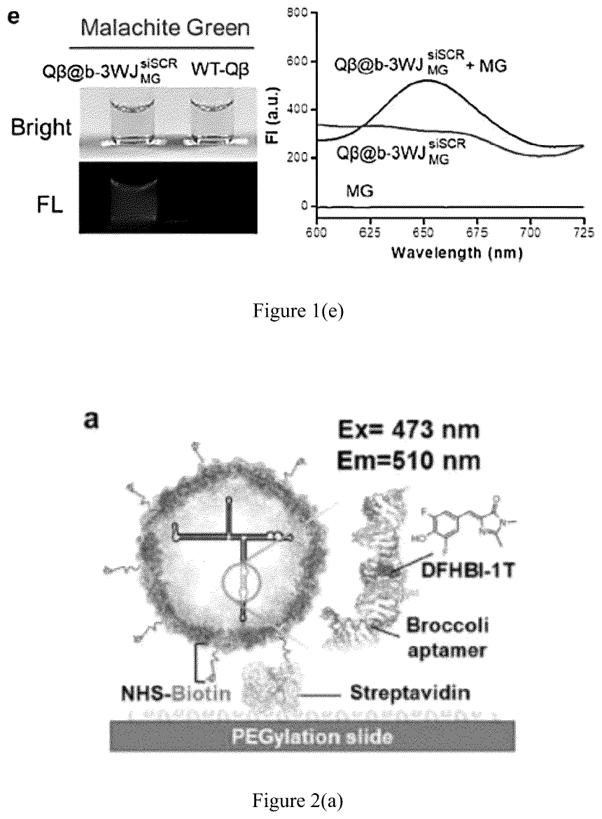

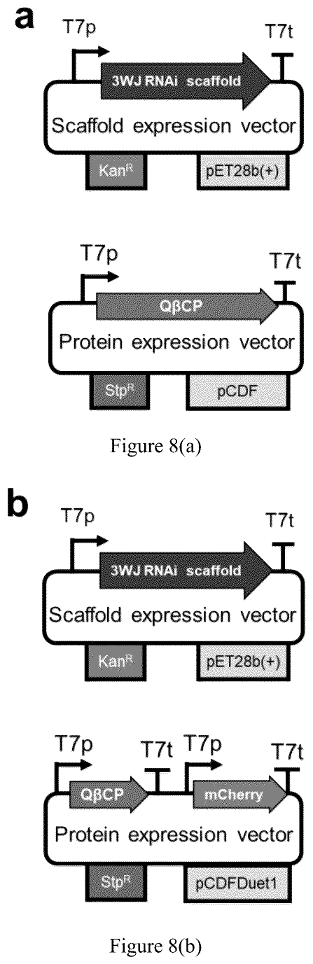

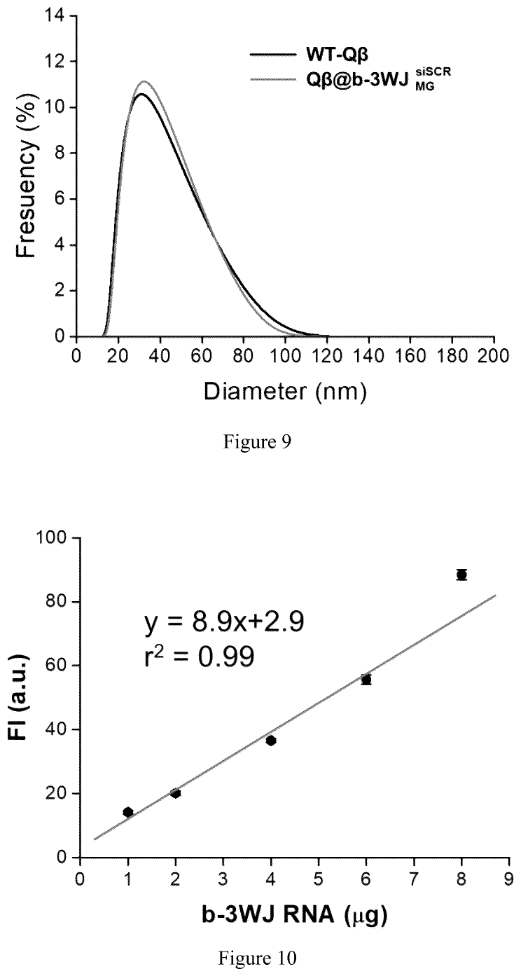

To impart better biosafety to the nucleic acid drug and considering that most manufacturing methods usually use liposomes to encapsulate naked RNA (i.e., siRNA, miRNA, mRNA), we designed a biological RNA production and selfpackaging system, including bacterial Qβ phage capsid, b-3WJ RNA scaffold production, and then automatic packaging. The b-3WJ RNA scaffold consists of the following parts: (1) bacterial Qβ phage capsid binding hairpin, (2) three functional regions junction together with a three-way junction motif from bacteriophage Phi29 hexameric motor pRNA. Such structures help to assist RNA stabilization and facilitate cellular ingestion resulting in therapeutic effects. To prove that the scaffold can be folded in the correct structure, we inserted scrambled siRNA (siSCR) (SEQ ID NO:6) at the left end, malachite green (MG) aptamer (SEQ ID NO:10) at the right end, and broccoli aptamer (SEQ ID NO:11) at the bottom of the scaffold, named b-3WJMG siSCR (FIG. 1(a)). The b-3WJsiSCR+MG and Qβ capsid were coexpressed in a dual-plasmid E. coli expression system (FIG. 8). After induction using IPTG, the transcribed b-3WJ MG siSCR with Qβ hairpin on the 5′ end and translated Qβ8 capsids self-assembled into Qβ VLPs with b-3WJ MG siSCR self-packaged inside, named Qβ@b-3WJsiSCR+MG. Packaging of the b-3WJ MG siSCR in the capsid left the morphology of the Qβ@b-3WJsiSCR+MG unchanged and showed uniform size distribution when compared with the wild-type Qβ VLPs (WT-Qβ) using transmission electron microscopy (TEM) (FIG. 1(b)); the diameter (Z-average) of Qβ@b-3WJsiSCR+MG was slightly increased to 30.6±0.4 nm from 30.4±0.2 nm (WT-Qβ) measured by dynamic light scattering (DLS), as shown in FIG. 9.

The results demonstrated that b-3WJsiSCR+MG encapsulation did not affect the self-assembly of the Qβ capsid. To confirm that b-3WJsiSCR+MG was packaged inside Qβ VLPs, we extracted RNA from Qβ3@b-3WJsiSCR+MG and performed Urea-PAGE electrophoresis, followed by staining with SY BR green and DFHBI-1T. A band at 237 nt was significantly observed only in the groups of IPTG induction and in vitro transcription in both PAGEs stained with SYBR green (FIG. 1c, left) and DFHBI-1T (FIG. 1(c), right). The results demonstrated that the b-3WJsiSCR+MG was successfully packaged inside, and the structure folded correctly because the broccoli aptamer in the scaffold reacted with DFHBI-1T to emit a green fluorescence band at 237 nt. Furthermore, the reactivity of Qβ@b-3WJsiSCR+MG with DFHBI-1T and MG was verified by incubating Qβ VLPs or Qβ@b-3WJsiSCR+MG with DFHBI-1T and MG solution. As shown in FIGS. 1d and 1e, only Qβ@b-3WJsiSCR+MG can react with DFHBI-1T and MG to exhibit green fluorescence (Em: 510 nm) and red fluorescence(Em: 652 nm), indicating that the packaged b-3WJsiSCR+MG can still react with the substrate to emit fluorescence for real-time cellular RNA process status monitoring. Thus, we can react the in vitro transcribed b-3WJ RNAs with DFHBI-1T to quantify the amount of b-3WJ RNAs packaged in Qβ VLPs by measuring the fluorescence intensity (FI) at 510 nm (FIG. 10). The results showed that the FI increased in proportion to the amount of b-3WJ RNAs.

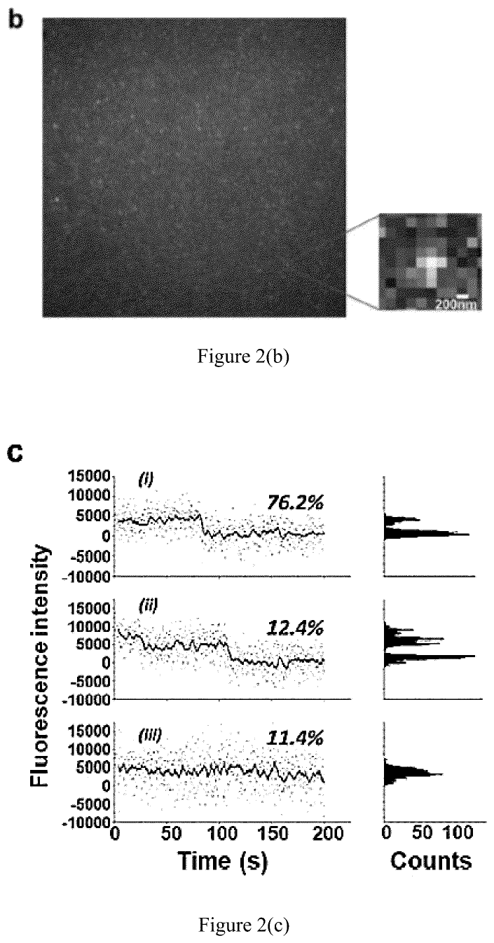

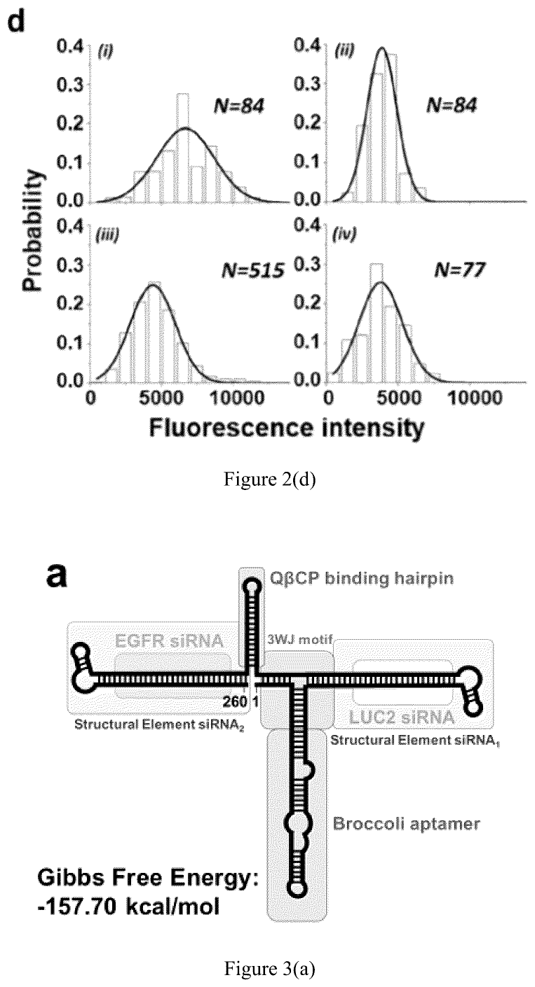

Several approaches have been developed to quantify a number of fluorophores, such as fluorescence fluctuation spectroscopy for moving complexes or localization fluorescence imaging systems, including photoactivated localization microscopy/stochastic optical reconstruction microscopy and single-molecule photobleaching (SMPB) imaging. To quantify the b-3WJ RNA molecules within individual Qβ VLPs, the SMPB system was adapted here. Individual Qβ@b-3WJsiSCR+MG were immobilized on polyethylene glycol (PEG)-ylated glass through a specific biotin-streptavidin interaction, and the fluorescence signals from DFHBI-1T/b-3WJ RNA complexes were acquired using a home-built TIRFM imaging system (FIG. 2a). Upon 473 nm excitation, fluorescence signals from individual Qβ@b-3WJsiSCR+MG were obtained. The fluorescence spot acquired and analyzed in our system exhibited a diffraction limit (FIG. 2b), representing the signal from individual Qβ@b-3WJsiSCR+MG. All acquired fluorescence intensity time traces can be classified into three different patterns: one-step photobleaching with a probability of 76.2%, two-step photobleaching with a probability of 12.4%, and no significant photobleaching during the acquisition time window with a probability of 11.4% (FIG. 2(c)). Averaged intensity values of 6872±1892 and 3889±1000 were obtained for the first-step and second-step photobleaching of individual Qβ@b-3WJsiSCR containing two DFHBI-1T/b-3WJ RNA complexes, respectively (FIG. 2(d)(i)-(ii)). For individual Qβ@b-3WJsiSCR+MG containing a single DFHBI-1T/b-3WJ RNA complex, an averaged intensity value of 4360±773, similar to the value obtained in the second-step photobleaching of Qβ@b-3WJsiSCR+MG containing two DFHBI-1T/b-3WJ RNA complexes, was obtained (FIG. 2(d) (iii)). For molecules exhibiting no significant photobleaching, an averaged intensity value of 3838±1400 was obtained, indicating the signal coming from a single DFHBI-1T/b-3WJ RNA complex in individual Qβ@b-3WJsiSCR+MG (FIG. 2(d)(iv)). Based on SMPB analysis, 87.6% of Qβ@b-3WJsiSCR+MG contains one DFHBI-1T/b-3WJ RNA complex in one Qβ VLP, while 12.4% of Qβ@b-3WJsiSCR+MG contains two DFHBI-1T/b-3WJ RNA complexes in one Qβ VLP.

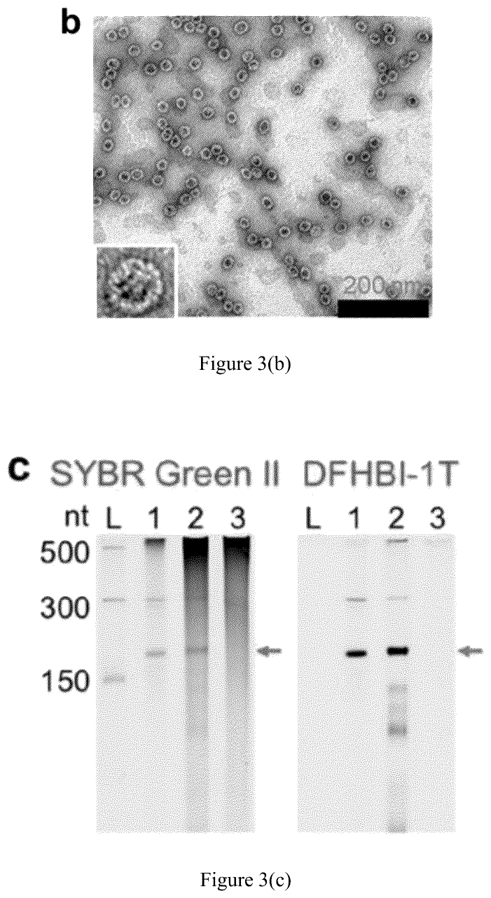

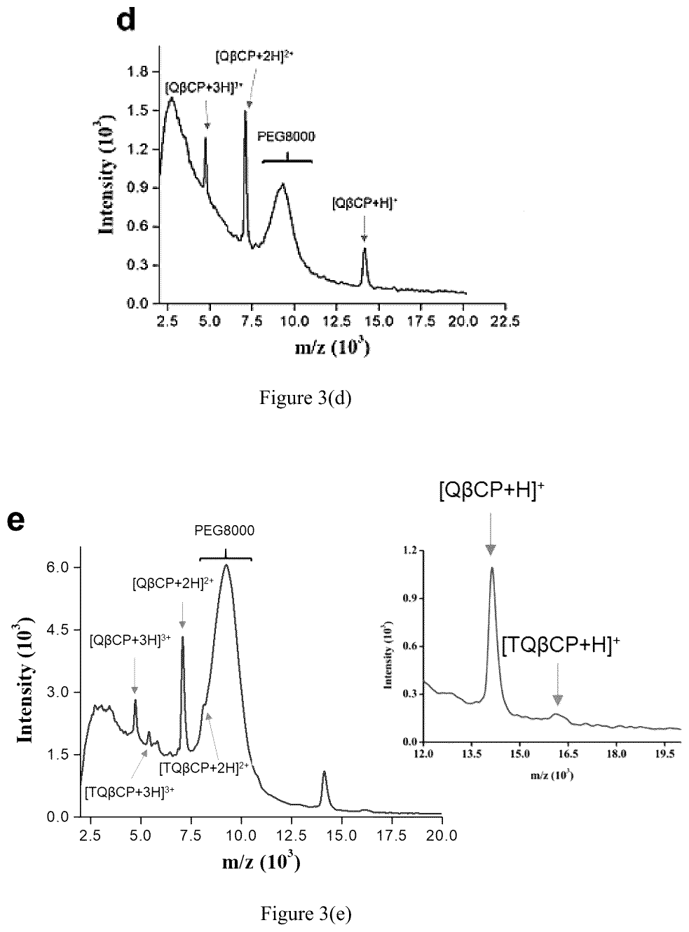

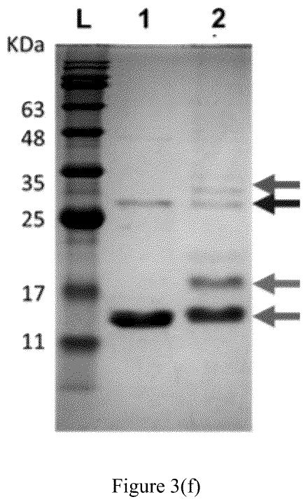

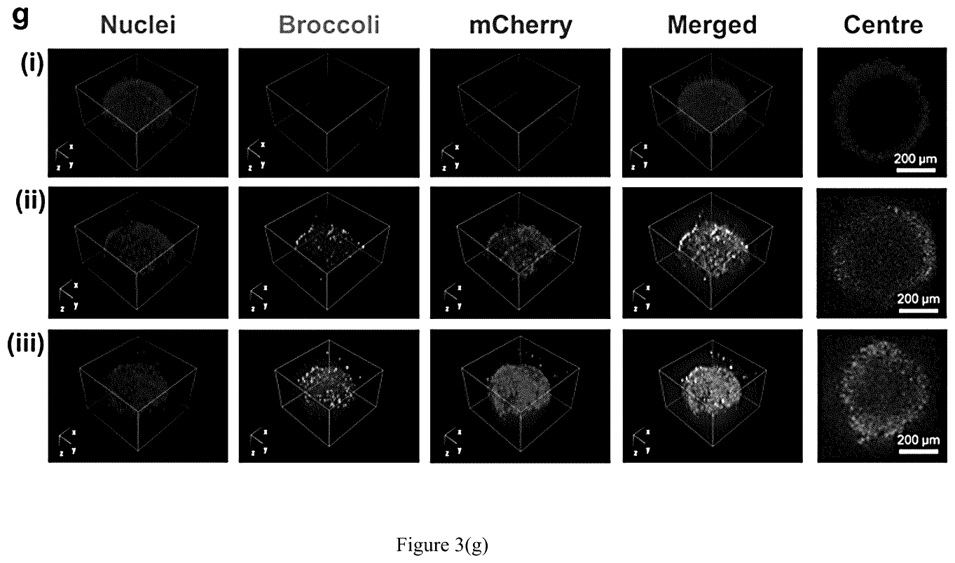

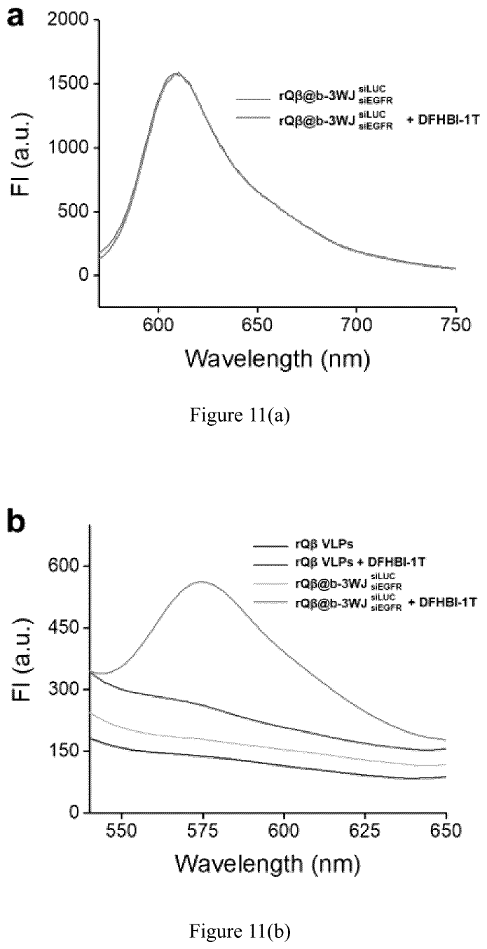

Next, we verified the effectiveness of Qβ@b-3WJ in gene silencing by replacing siSCR with EGFR siRNA (siEGFR) (SEQ ID NO:7) and MG aptamer (SEQ ID NO:10) with luciferase siRNA (siLUC) (SEQ ID NO:8) in the 3WJ RNA scaffold, named rQβ@b-3WJsiEGFR+siLUC (FIG. 3(a) and FIG. 8(b)). After IPTG induction and purification, the extracted rQβ@b-3WJsiEGFR+siLUC showed a uniform spherical shape with a diameter of approximately 31.4±0.5 nm, proving again that mCherry protein and b-3WJsiEGFR+siLUC packaging do not affect the selfassembly of Qβ phage capsids (FIG. 3(b)). SYBR green II- and DFHBI-1T-stained urea-polyacrylamide gel electrophoresis (PAGE) also confirmed that b-3WJsiEGFR+siLUC was indeed packaged inside Qβ VLPs by observing the band at 260 nt only in the in vitro transcription and rQβ@b-3WJsiEGFR+siLUC groups (FIG. 3(c)). Furthermore, rQβ@b-3WJsiEGFR+siLUC was suspended in D FH BI-1T solution, and a significant fluorescence peak at 611 nm was obtained upon 473 nm excitation (FIGS. 11(a) and 22(b)), indicating that the copackaged mCherry protein would not interfere with the reactivity of broccoli aptamer with DFHBI-1T. However, the emission of DFHBI-1T shifted to 575 nm from 510 nm, most likely because the DFHBI-1T (donor) emission spectrum overlaps with the mCherry (acceptor) excitation spectrum to excite the packaged mCherry. The results indicated that the mCherry protein and b-3WJsiLUC siEGFR were successfully produced by E. coli and copackaged in Qβ VLPs.

Furthermore, the TAT peptide was modified on the surface of rQβ@b-3WJsiEGFR+siLUC, named TrQβ@b-3WJsiEGFR+siLUC, to enhance cellular internalization, most likely because the TAT peptide originates from the HIV transactivator of transcription protein, which has demonstrated excellent potential in translocating across the plasma membrane of various cell types. Successful TAT peptide conjugation was confirmed by Matrix-assisted laser desorption ionization-time-of-flight mass spectrometry (MALDI-TOFMS) and sodium dodecyl sulfate (SDS)-PAGE. The representative MALDI-TOFMS spectra of rQβ@b-3WJsiEGFR+siLUC and TrQβ@b-3WJsiEGFR+siLUC are shown in FIGS. 3(d) and 3(e). For TrQβ@b-3WJsiEGFR+siLUC, new peaks appeared at m/z 5390, m/z 8095, and m/z 16505 VLPs belong to one 3H+ionized TAT conjugated Q/3 capsid protein (TQβCP), 2H+ionized TQβCP, and H+ionized TQβCP compared with Qβ@b-3WJsiLUC siEGFR, indicating that TAT peptides were indeed conjugated on the surface of Qβ@b-3WJsiLUC siEGFR. The results were also confirmed by SDS-PAG E, and new bands appeared at 17.1 kDa (single TAT peptide conjugation on one Qβ capsid protein monomer) and 19.8 kDa (two TAT peptides conjugated on one Qβ capsid protein monomer) after TAT peptide conjugation (FIG. 3(f), Lane 2) compared with nonmodified rQβ@b-3WJsiEGFR+siLUC (FIG. 3(f), Lane 1), thereby proving the successful conjugation of TAT peptides on rQβ@b-3WJsiEGFR+siLUC to form TrQβ@b-3WJsiEGFR+siLUC. Subsequently, we incubated the U87MG cells with rQβ@b-3WJsiEGFR+siLUC and TrQβ@b-3WJsiEGFR+siLUC to compare their ability of getting into the cells. The distributions of TrQβ VLPs (red fluorescence) and b-3WJsiEGFR+siLUC (green fluorescence) were both markedly higher in the whole tumor spheroid and can penetrate deeper into the tumor spheroid than in the absence of TAT peptide modified rQβ@b-3WJsiEGFR+siLUC (FIG. 3(g)), indicating that TAT peptides can indeed effectively increase tumor cell uptake and penetration. This enhanced adsorption and transportation was essential for inhibiting tumor growth.

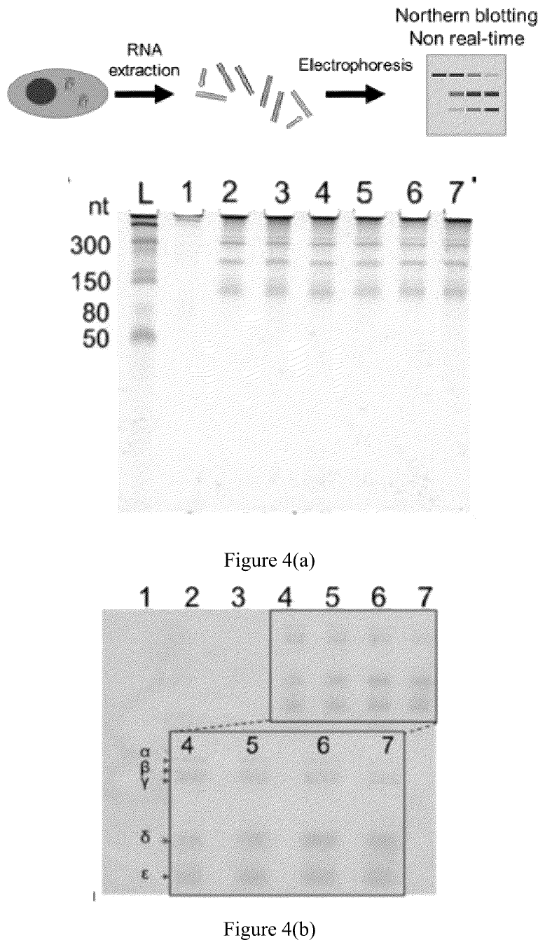

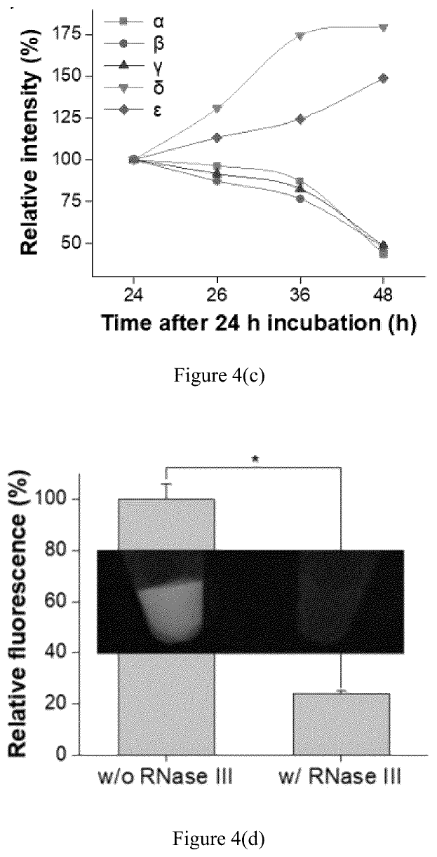

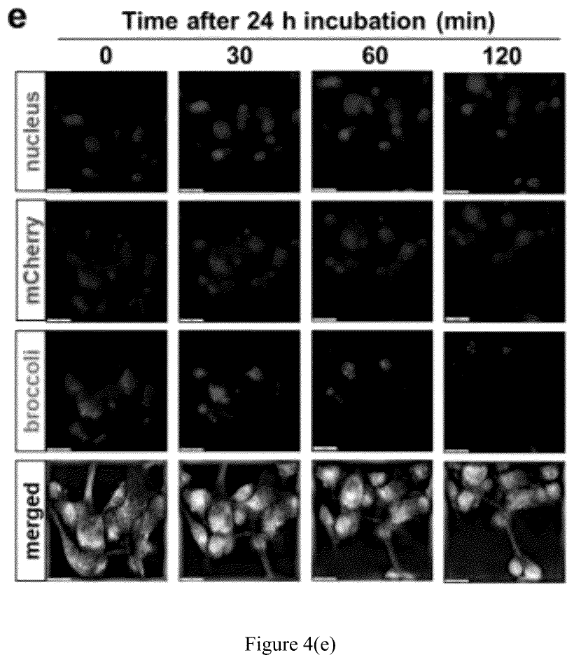

When the RNA duplex is delivered into the cells, the ribonuclease (RNase) III enzyme Dicer processes double stranded RNAs (dsRNAs) into 21-22-nt-long duplexes with 2-nt 3′overhangs, guiding sequence-specific degradation of complementary mRNAs (mRNAs) once incorporated into the RNA-induced silencing complex (RISC). Dicer-2 (Dcr-2) processes long double-stranded precursors and generates siRNAs, while Dicer-1 (Dcr-1) processes pre-mi RNAs into mature mi RNAs. To date, Northern blotting is a promising tool to observe the intracellular cleavage status of RNA with Dicer enzyme. In this study, to determine when the delivered b-3WJsiLUC siEGFR is cleaved in cells by Dicer enzyme, the extracted RNAs from cells with different treatments were analyzed by Northern blotting. From 10% Urea-PAGE stained with SY BR green II, the RNA signal was detected in all cell samples with different treatments (FIG. 4(a), Lanes 2-7). Using a biotinconjugated DNA probe complementary to the siEGFR strand of b-3WJsiEGFR+siLUC, only the samples from the cells treated with TrQβ@b-3WJsiEGFR+siLUC still exhibited an obvious RNA signal (FIG. 4b, Lanes 4-7), indicating that TrQβ@b-3WJsiEGFR+siLUC can be internalized into cells and release b-3WJsiLUC siEGFR. Furthermore, the delivered b-3WJsiEGFR+siLUC began to be cleaved into small fragments as the incubation time increased. By comparison to the sample from the cells incubated with TrQβ@b-3WJsiEGFR+siLUC for 24 h (FIG. 4(b), Lane 4), the large fragments of b-3WJsiEGFR+siLUC significantly decreased, while the small fragments significantly increased after an additional 48 h of incubation (FIG. 4(b), Lane 7 and FIG. 4c), indicating that the delivered b-3WJsiEGFR+siLUC could be released from TrQβ@b-3WJsiEGFR+siLUC and then cleaved by the Dicer enzyme to specifically degrade the complementary mRNAs. However, Northern blotting is not only complicated and time-consuming but also cannot monitor the RNA cleavage status in real time. Otherwise, our designed b-3WJsiEGFR+siLUC can react with DFHBI-1T to emit green fluorescence when its structure is intact, but the broccoli aptamer loses its reactivity with DFHBI-1T when the b-3WJsiEGFR+siLUC is cleaved by Dicer enzyme, resulting in a decrease in green fluorescence. As proof, we treated thein vitro transcribed b-3WJsiEGFR+siLUC with a ShortCut RNase III kit at 37° C. for 20 min, and the green fluorescence was significantly weakened compared to the green fluorescence of the nontreated b-3WJsiEGFR+siLUC (FIG. 4(d)), indicating that b-3WJsiEGFR+siLUC can be cleaved into small fragments by the Dicer enzyme and lose its reactivity with DFHBI-1T. Subsequent incubation of TrQβ@b-3WJsiEGFR+siLUC with U87MG cells in the presence of DFHBI-1T allowed us to monitor the distribution of TrQβ@b-3WJsiEGFR+siLUC and the cleavage status of b-3WJsiEGFR+siLUC simultaneously in cells using a fluorescence microscope. As shown in FIG. 4(e), a large amount of TrQβ8@b-3WJsiEGFR+siLUC was endocytosed into U87MG cells to exhibit strong red fluorescence (TrQβ VLPs) and green fluorescence (b-3WJsiEGFR+siLUC) after 24 h of incubation, indicating the b-3WJsiEGFR+siLUC can be indeed delivered into tumor cells. The green fluorescence in cells gradually disappeared with increasing incubation time, while the intracellular red fluorescence did not decrease at all. The results indicated that b-3WJsiEGFR+siLUC can be successfully delivered into cells by TrQβ VLPs and that its cleavage status with the Dicer enzyme can easily be monitored by fluorescence microscopy in real time.

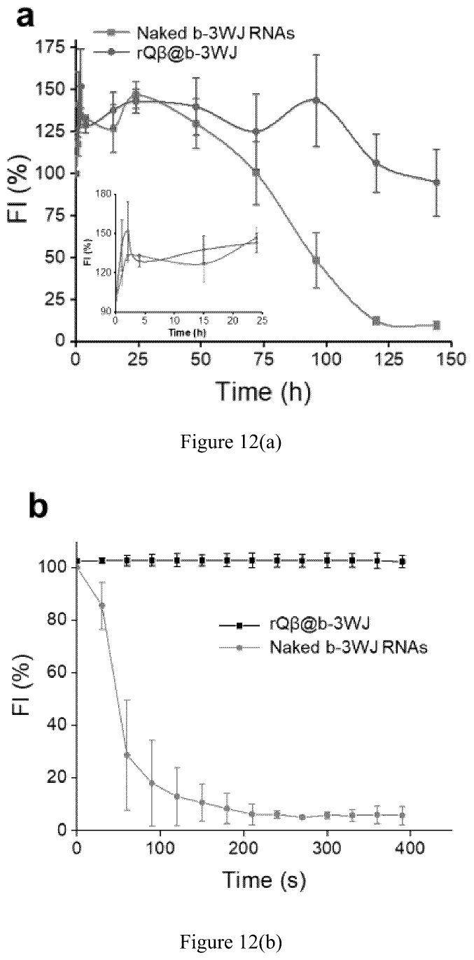

RNA stability is one of the important factors affecting the success or failure of RNA interference (R N A i) therapy. However, packaging in rQβ VLPs protects b-3WJsiEGFR+siLUC against urea-mediated denaturation and RNase A-mediated cleavage. We incubated rQβ@b-3WJsiEGFR+siLUC with urea and RNase A and then mixed it with DFHBI-1T to assess the integrity of the b-3WJsiEGFR+siLUC structure. After 144 h of incubation in the biological urea concentration (10 mM) urea at 37° C., the b-3WJsiEGFR+siLUC within rQβ VLPs was approximately 80% intact. In contrast, less than 10% of naked b-3WJsiEGFR+siLUC was intact under the same conditions (FIG. 12(a)). Similarly, nearly 100% of rQβ VLPs packaged with b-3WJsiEGFR+siLUC remained intact after 400 s at an RNase A concentration of 0.1 mg/mL, most likely because rQβ VLPs protect packaged b-3WJsiEGFR+siLUC from degradation by nucleases, which are larger than the pore size of the rQβ VLPs. There was no detectable intact b-3WJsiEGFR+siLUC remaining after the same incubation time for naked b-3WJsiEGFR+siLUC under the same conditions (FIG. 12(b)). Taken together, the results show that packaged RNA in rQβ VLPs degrades slowly at 37° C. in the presence of urea and RNase A without complex structural modifications that has considerable potential for the development of nucleic acid drugs.

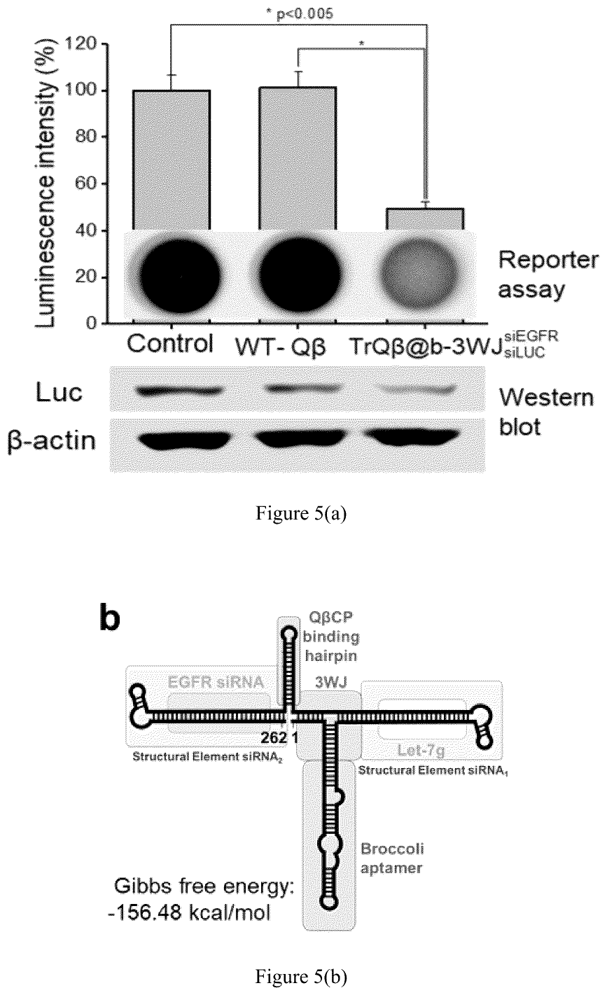

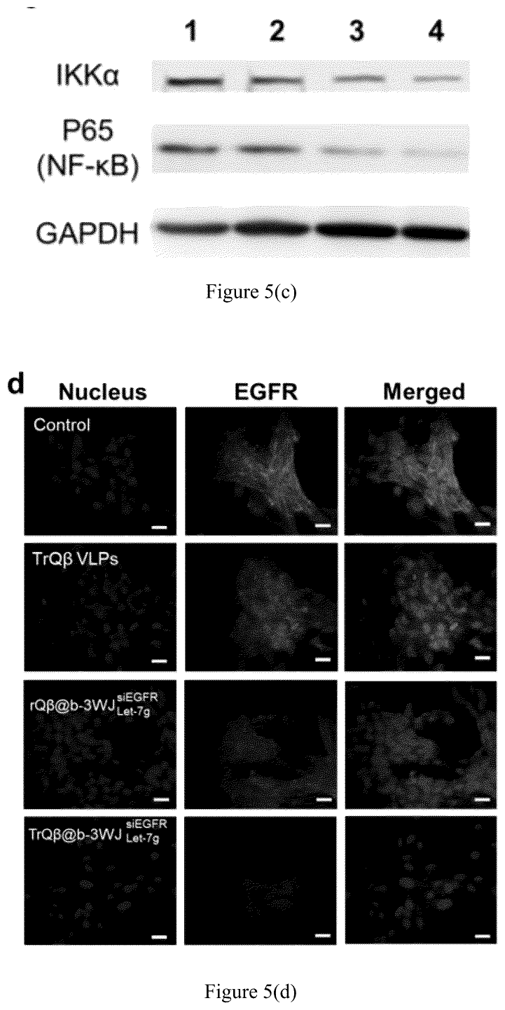

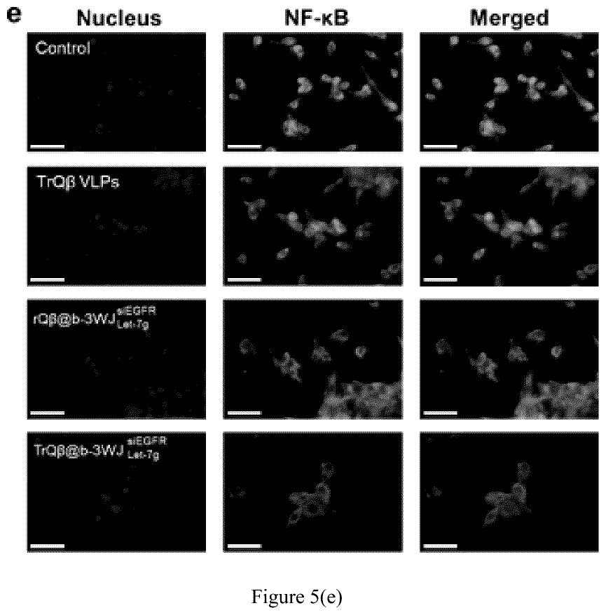

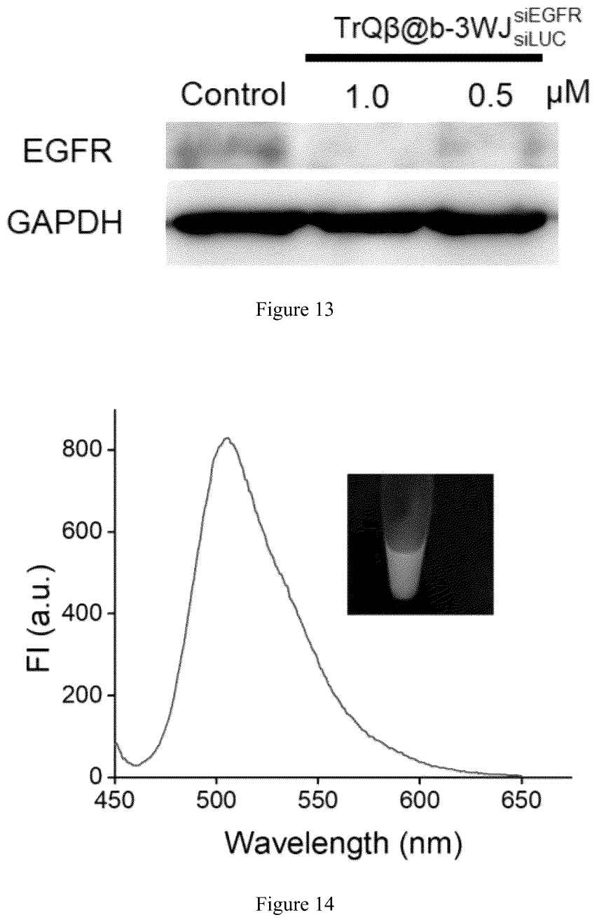

Next, we assessed the gene silencing ability of TrQβ@b-3WJsiEGFR+siLUC to EGFR and luciferase in luciferase-expressing U87MG cells. Incubation of luciferase-expressing U87MG cells with TrQβ@b-3WJsiEGFR+siLUC decreased the expression of luciferase, as shown by bioluminescence imaging and Western blot analysis. The results show that incubation in 1 μM TrQβ@b-3WJsiEGFR+siLUC for 72 h efficiently knocked down approximately 55% of luciferase expression compared with the control and WT-Qβ-treated groups (FIG. 5(a)). Moreover, TrQβ@b-3WJsiEGFR+siLUC also showed excellent inhibition of EGFR expression, and approximately 91% expression of EGFR was suppressed in U87MG cells compared with the control group (FIG. 13). The overall results indicate that inhibition is caused by delivered b-3WJsiEGFR+siLUC, but not by TrQββ VLPs, and the TrQβ@b-3WJsiEGFR+siLUC can simultaneously downregulate two target genes.

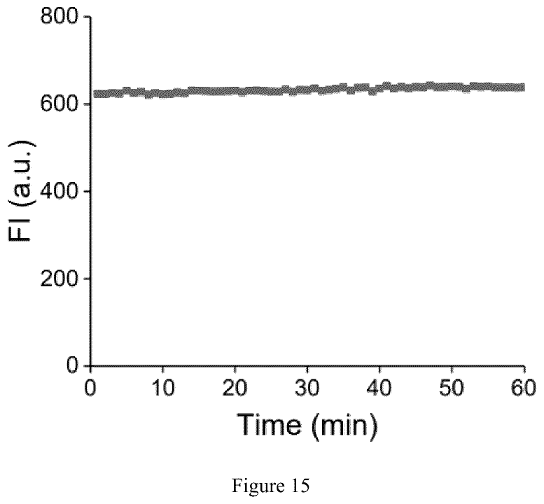

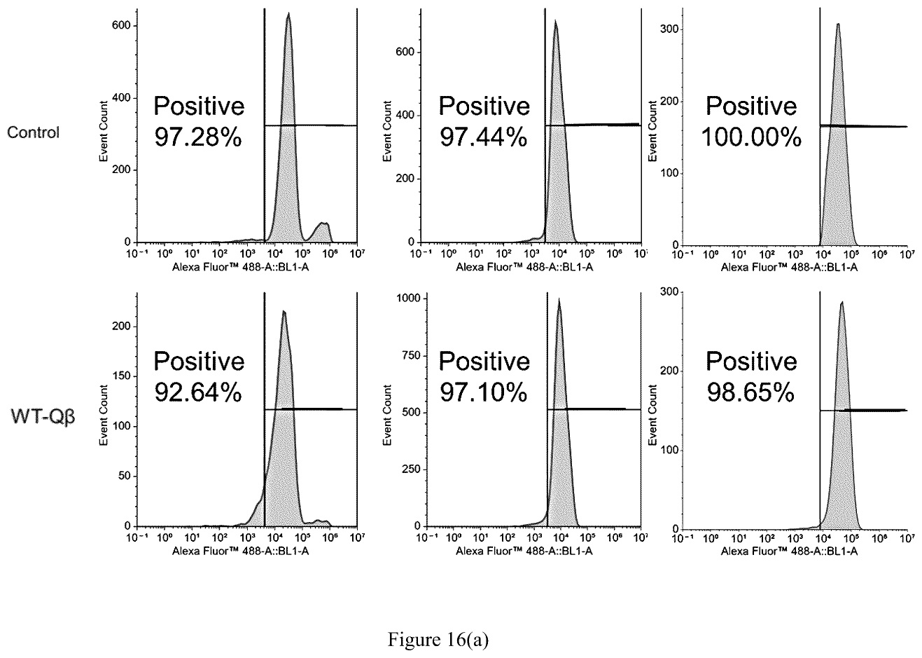

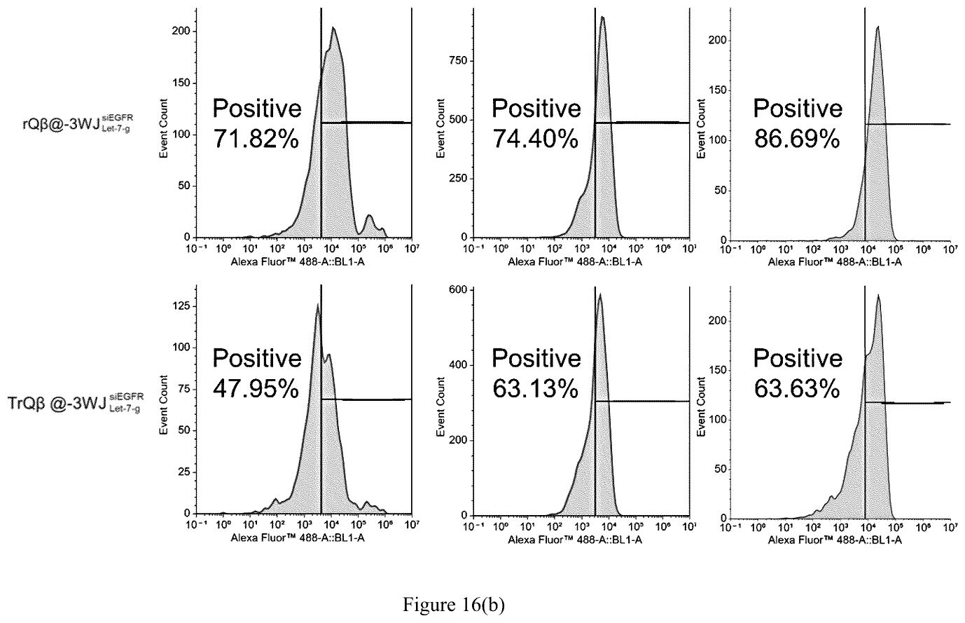

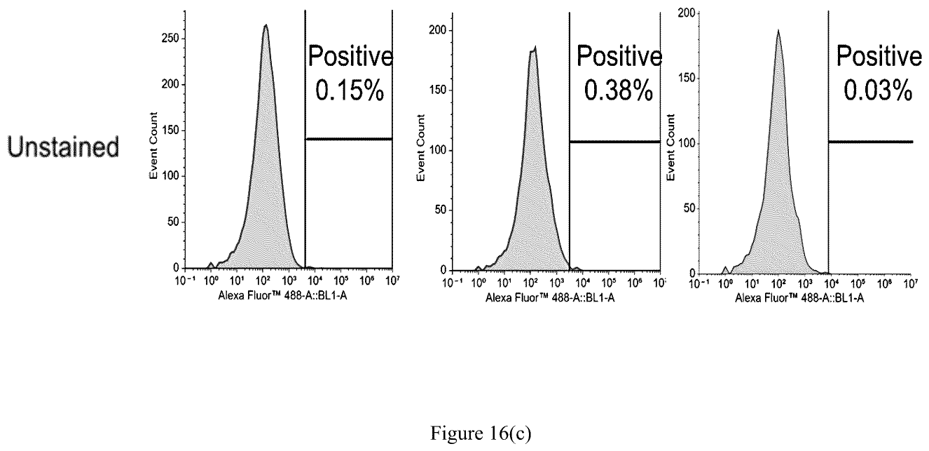

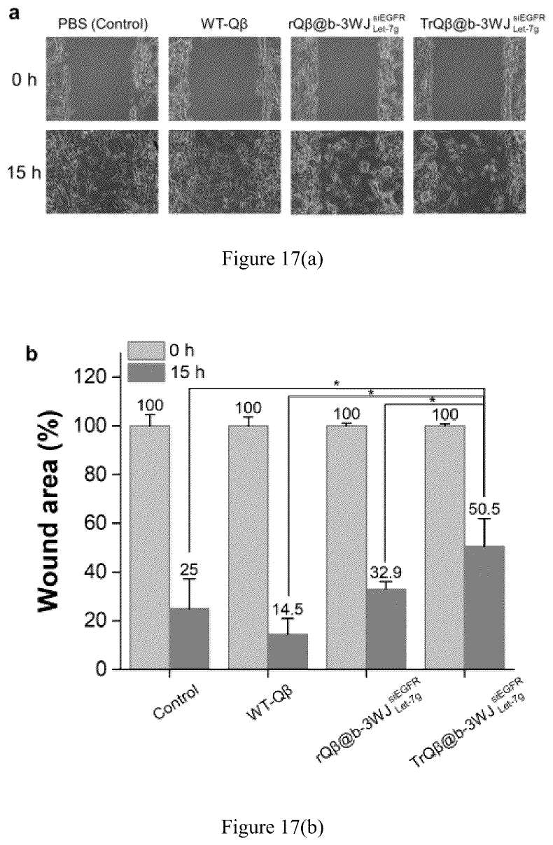

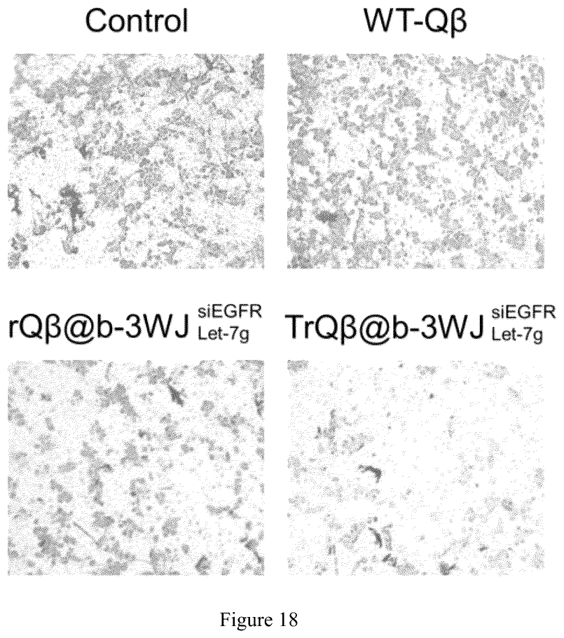

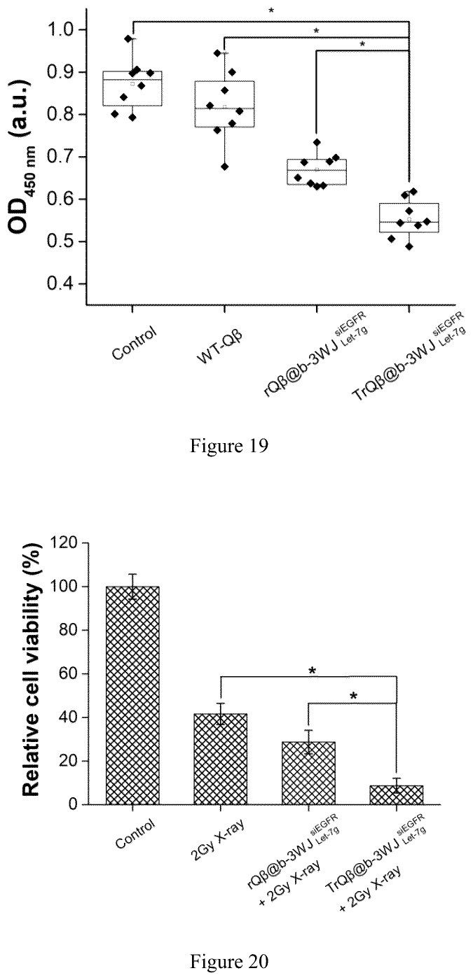

Nuclear factor kappa B (NF-κB) is well established to be activated in response to a variety of DNA lesions, such as TMZ-induced SN 1 methylation, cisplatin-induced DNA crosslinking, and IR-induced double-strand breaks (DSBs). Inhibition of NF-κB increases the sensitivity of cancer cells to the apoptotic action of chemotherapeutic agents and radiation exposure. In humans, the IκB kinase (IKK) complex serves as the master regulator for the activation of NF-κB by various stimuli. The IKK complex consists of IKKα and IKKβ, which form the catalytic subunit, and the regulatory subunit IKKγ. Previous reports pointed out that the downregulation of EGFR expression would affect RIPK1/TAK-1/TRAF2 message transmission and thus inhibit the expression of the IKK complex. Let-7g cleaves IKKα mRNA and may play an important role in the response to IR through the inhibition of NF-κB. Therefore, we replaced siLUC with Let-7 g miRNA (SEQ ID NO:9) in the 3WJ RNA scaffold and performed the production, named rQβ@b-3WJsiEGFR+Let-7g (FIG. 5b). The obtained rQβ@b-3WJsiEGFR+Let-7g modified with TAT peptides on the surface (TrQβ@b-3WJsiEGFR+Let-7g) can still emit green fluorescence in the presence of DFHBI-1T, indicating that the structure of b-3WJsiEGFR+Let-7g still folded correctly after sequence exchange (FIG. 14) and could be protected from RNase A degradation for more than 1 h (FIG. 15). To verify the effectiveness of TrQβ@b-3WJsiEGFR+Let-7g in the downregulation of NF-κB for enhanced radiotherapy, the expression of EGFR and NF-κB and the translocation of NF-κB to the nucleus were confirmed by Western blotting and immunofluorescence staining. The results show that both rQβ@b-3WJsiEGFR+Let-7g and TrQβ@b-3WJsiEGFR+Let-7g can downregulate the expression of IKKα and NF-κB (p65) compared with control and TrQβ VLPs groups. However, the TrQβ@b-3WJsiEGFR+Let-7g showed higher gene silencing efficiency for IKKα and NF-κB (P65) than rQβ@b-3WJ Let-7g si EGFR because the TA T peptides on TrQβ@b-3WJsiEGFR+Let-7g effectively increase tumor cell uptake and penetration. Subsequently, the gene silencing efficiency of TrQβ@b-3WJsiEGFR+Let-7g for EGFR and NF-κB in U87MG cells was also confirmed by immunofluorescence staining. The EGFR expression (green fluorescence) on cell membrane was significantly downregulated by TrQβ@b-3WJsiEGFR+Let-7g and much lower than that by rQβ@b-3WJsiEGFR+Let-7g (FIG. 5d). Not only that, the translocation of NF-κB to the nucleus of U87MG cells was also significantly inhibited (no green fluorescence exhibited in nucleus) after treatment with TrQβ@b-3WJsiEGFR+Let-7g compared with control (PBS), TrQβ VLPs, and rQβ@b-3WJsiEGFR+Let-7g treated groups (FIG. 5(e)). We then quantified the gene silencing efficiency of rQβ@b-3WJsiEGFR+Let-7g and TrQβ@b-3WJsiEGFR+Let-7g for EGFR, IKKα, and NF-κB in U87MG cells using flow cytometry (FIGS. 16(a) to 16(c)). The results showed that the TrQβ@b-3WJsiEGFR+Let-7g could inhibit approximately 52.05% of EGFR protein expression (28.18% for rQβ@b-3WJsiEGFR+Let-7g), 36.87% of IKKα protein expression (25.60% for rQβ@b-3WJsiEGFR+Let-7g), and 36.37% of NF-κB protein expression (13.31% for rQβ@b-3WJsiEGFR+Let-7g) compared with the control group. These results indicating that TrQβ@b-3WJsiEGFR+Let-7g can deliver more b-3WJsiEGFR+Let-7g than rQβ@b-3WJsiEGFR+Let-7g into U87MG cells to significantly enhance the efficiency of radiotherapy by effectively downregulating the expression of NF-κB because the overexpression of NF-κB promotes DNA repair. In addition, inhibition of EGFR and IKK complex expression by TrQβ@b-3WJsiEGFR+Let-7g not only inhibited DNA repair but also effectively suppressed cell migration by 40-50% in a wound healing assay (FIGS. 17(a) and 17(b)) and inhibited the invasion rate by approximately 60% in a trans-well assay (FIG. 18). The results were also confirmed by observing cell progression after pretreatment with TrQβ@b-3WJsiEGFR+Let-7g, showing that the cell progression rate of pretreated U87MG cells was significantly slower than the cell projection rate of the control group (without any treatment), WT-Qβ, and rQβ@b-3WJsiEGFR+Let-7g treatment groups (FIG. 19). According to the above results, TrQβ@b-3WJsiEGFR+Let-7g (with TAT peptide conjugation) have been proven to have superior cell penetration efficiency than rQβ@b-3WJsiEGFR+Let-7g (without TAT peptide conjugation). Therefore, we then investigated the in vitro synergistic effect of EGFR/IKKα gene silencing and X-ray irradiation (FIG. 20). The cell viability of U87MG cells through the pretreatment with TrQβ@b-3WJsiEGFR+Let-7g for 72 h followed by 2Gy X-ray irradiation was decreased to 8.7±3.4% compared with the 2Gy X-ray irradiated group (cell viability: 41.6±4.9%) and rQβ@b-3WJsiEGFR+Let-7g and 2Gy X-ray irradiated group (cell viability: 28.7±5.4%). These findings indicate that the rQβ@b-3WJsiEGFR+Let-7g can be efficiently delivered by TrQβ VLPs to perform dual-target gene (EGFR and IKKα) silencing for further radiotherapy enhancement in GBMs.

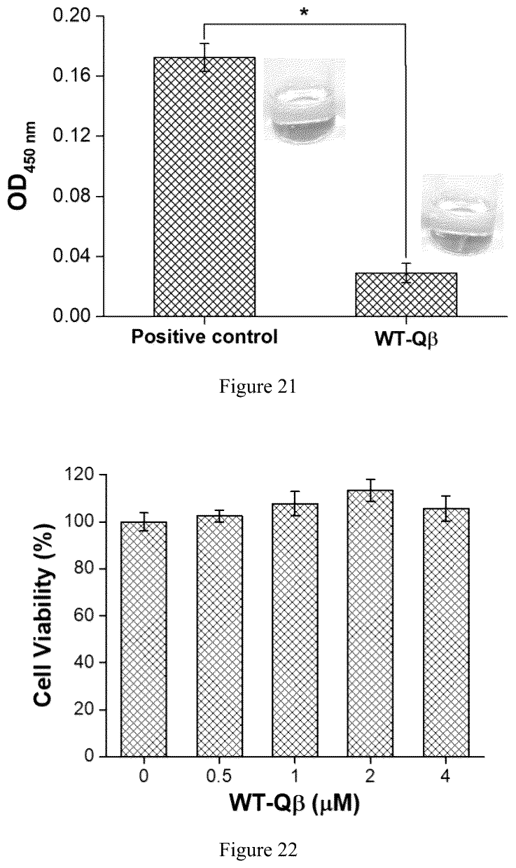

In order to ensure that the Qβ VLPs-based therapeutics prepared the bioproduction process are not toxic to mice, we analyzed the endotoxin concentration in WT-Qβ. The results showed that the purified WT-Qβ are almost free of endotoxin (FIG. 21). We also investigated the cytotoxicity toward U87MG cells induced by WT-Qβ (without 3WJ RNAs). No significant inhibition of cell proliferation was observed in the WT-Qβ treated group (from 0.5 to 4 μM) for 24 h compared with control group (FIG. 22). The overall results indicate that Qβ VLPs-based therapeutics are safe enough to be used in future clinical gene therapy. The promising dual-gene silencing and RNAi distribution monitoring outcomes instigated our exploration into whether TrQβ@b-3WJsiEGFR+Let-7g performed a therapeutic response well to tumors in vivo.

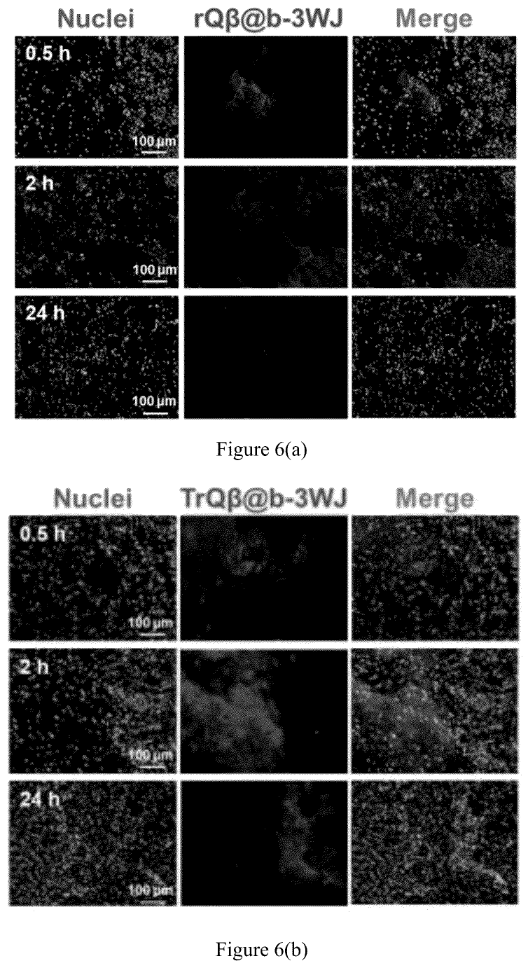

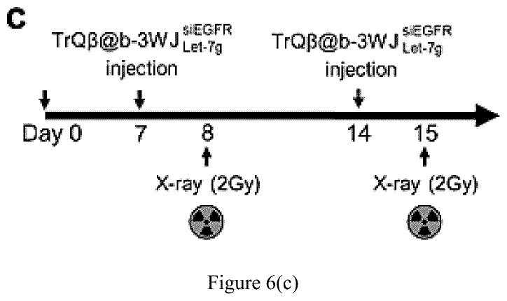

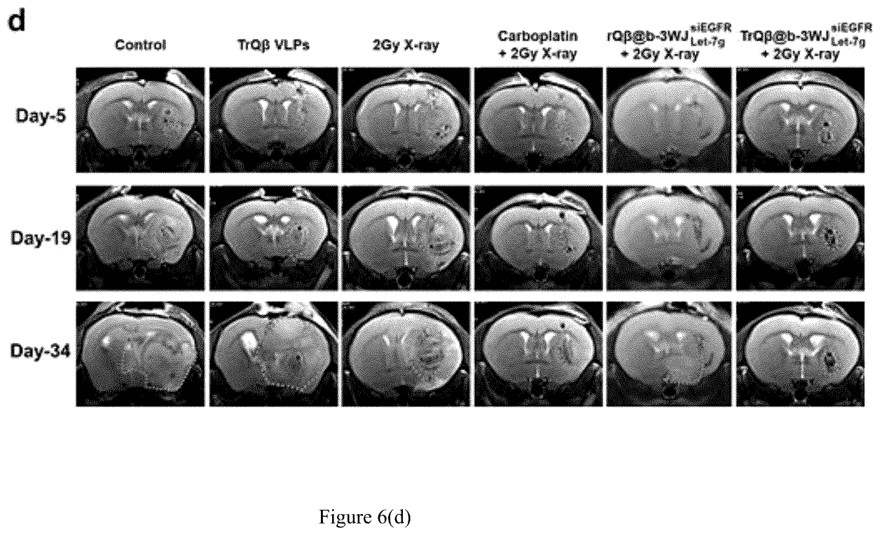

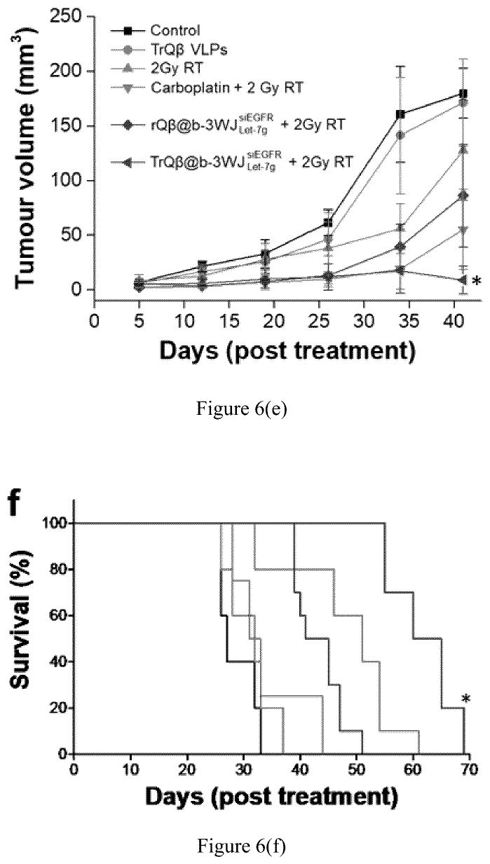

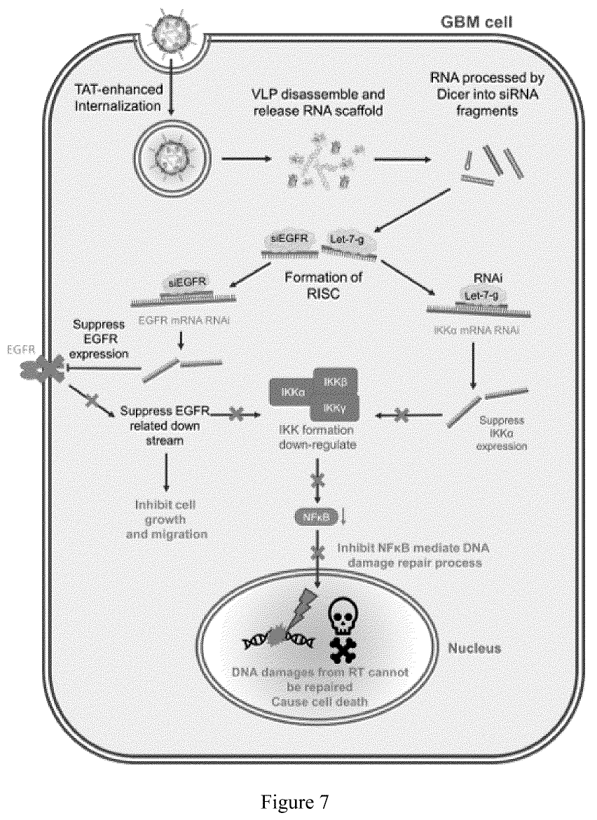

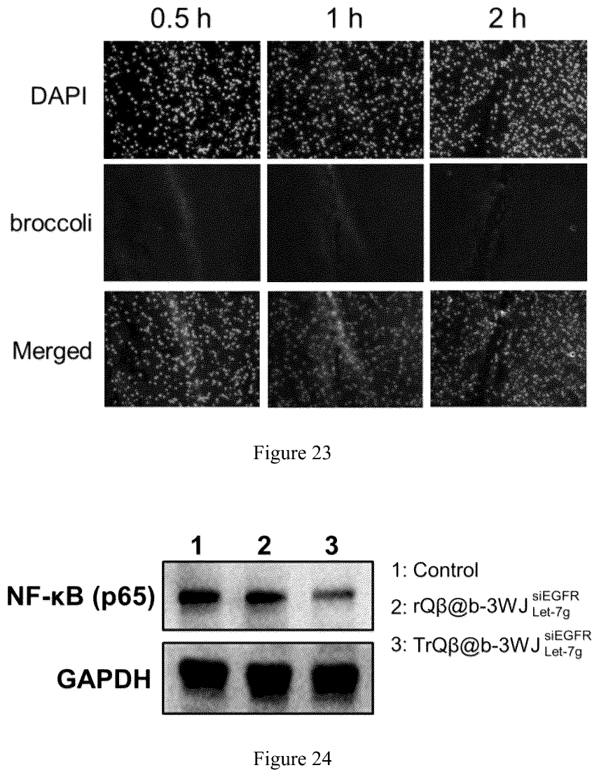

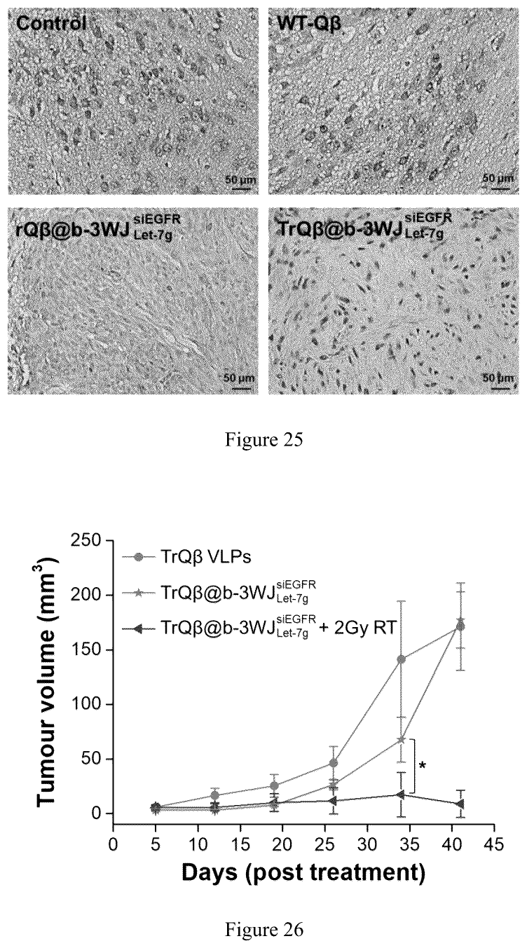

Next, we investigated the distribution of the Qβ VLPs-based therapeutics in tumor tissue after CED infusion (FIG. 6(a) and 6(b)). Both rQβ@b-3WJsiEGFR+Let-7g and TrQβ@b-3WJsiEGFR+Let-7g can penetrate into the tumor tissue from the injection site located at the junction of tumor tissue and normal brain tissue after 0.5 h of CED infusion and reached the maximum dose at 2 h. Moreover, increasingly stronger red fluorescence signals were observed for TrQβ@b-3WJ Let-7g siEGFR and significantly retained inside tumor tissue for a longer time and have not been eliminated until 24 h after CED infusion, indicating excellent tumor penetrability. Furthermore, b-3WJ Let-7g siEGFR was taken up in mouse brain tumor cells and was easily monitored without extra tracer labeling after administration of TrQβ@b-3WJsiEGFR+Let-7g by CED using a fluorescence microscope. As shown in FIG. 23, the green fluorescence accumulated at the injection site in the mouse brain tumor clearly after administration of TrQβ@b-3WJsiEGFR+Let-7g by CED. Moreover, the delivered b-3WJsiEGFR+Let-7g began to spread from the injection site to surrounding tissues and entered cells 2 h after administration for efficient gene silencing. The above-mentioned promising in vitro outcomes instigated our exploration into whether TrQβ@b-3WJsiEGFR+Let-7g performed well in vivo. In the first, we investigated the gene silencing efficiency of rQβ@b-3WJsiEGFR+Let-7g and TrQβ@b-3WJsiEGFR+Let-7g to downregulate the expression of NF-κB by Western blot analysis and immunohistochemistry. The results demonstrated that the concentration of NF-κB in tumor tissue cells can be reduced when the tumor-bearing mice received one dose of rQβ@b-3WJsiEGFR+Let-7g, and reduced more when the mice received one dose of TrQβ@b-3WJsiEGFR+Let-7g (FIGS. 24 and 25). Subsequently, the tumor-bearing mice, through transplantation of U87MG cells into their brains, were infused with 5 μL of carboplatin or rQβ@b-3WJsiEGFR+Let-7g or TrQβ@b-3WJsiEGFR+Let-7g by CED infusion followed by 2Gy X-ray irradiation (FIG. 6c). The above course of treatment was repeated after 7 days of initial treatment. Brain magnetic resonance (MR) images were obtained from each animal subgroup to measure the brain tumor volume after various treatments (FIG. 6(d)). The effect of EGFR and IKKα dual-gene silencing and thereby NF-κB downregulation by TrQβ@b-3WJsiEGFR+Let-7g on tumor progression with low-dose X-ray irradiation (2Gy) was analyzed, and the results are presented in FIGS. 6e and f. The tumor-bearing mice without any treatment (control; 160.5±44.0 mm3 on Day 34 and 179.7±22.8 mm3 on Day 41) and the mice that received two doses of TrQβ VLPs via CED infusion (141.3±53.2 mm³ on Day 34 and 171.3±40.1 mm³ on Day 41) all developed large brain tumors in the treated hemisphere, resulting in a median survival time of 27 days and 32 days for the control and TrQβ VLPs-treated groups, respectively. Although the tumor-bearing mice that received two rounds of 2Gy X-ray irradiation or two doses of TrQβ@b-3WJsiEGFR+Let-7g via CED infusion all developed significantly smaller brain tumors (56.0±22.7 mm3 for X-ray irradiation only and 67.7±20.7 mm3 for TrQβ@b-3WJsiEGFR+Let-7g only on Day 34) than the control and TrQβ VLPs-treated groups, recurrence was observed after 34 days of treatment (127.5±45.8 mm3 for Xray irradiation only and 177.3±25.7 mm3 for TrQβ@b-3WJsiEGFR+Let-7g only on Day 41). No obvious improvement in survival rate was observed (median survival=31 days for both groups) compared with the control and TrQβ VLPs-treated groups by the log-rank analysis (Table Si), indicating that (1) the 2Gy X-ray may not cause enough double-strand breaks (DSBs) and that the cells still process DNA repair to promote tumor cell growth, (2) the TrQβ@b-3WJsiEGFR+Let-7g can only perform gene silencing to inhibit the DNA repair and slow down the tumor cell growth rate, not directly kill tumor cells (FIG. 26). Significant tumor growth inhibition was observed (18.9±17.8 mm3 on Day 34) compared with the 2Gy X-ray irradiation group when the mice received 5 PL of carboplatin via CED infusion followed by 2Gy X-ray irradiation, most likely because carboplatin is known to sensitize cells to X-rays by enhancing the generation of DSBs and persistent single-strand breaks (SSBs). However, slight recurrence was observed after 34 days of treatment (55.2±36.5 mm3 on Day 41), most likely because carboplatin has only the function of enhancing the generation of DSBs without the ability of DNA repair inhibition. Only one of the ten mice (10%) that received CED infusion of two doses of carboplatin at a volume of 5 μL for each followed by 2Gy X-ray irradiation survived over 54 days (median survival=51 days). Even so, its therapeutic efficiency is still better than that of rQβ@b-3WJsiEGFR+Let-7g enhanced radiotherapy group (median survival=41 days) due to the relatively low cell penetration efficiency of rQβ@b-3WJsiEGFR+Let-7g, which could not efficiently downregulate NF-κB expression to inhibit DNA repair. Noteworthy, the combination of TrQβ@b-3WJsiEGFR+Let-7g pretreatment with 2Gy Xray irradiation provided nearly complete suppression of tumor progression (8.9±12.5 mm3 on Day 41, and no significant tumor recurrence was observed until Day 50 using this gene silencing-enhanced radiotherapy), which resulted in approximately 50% treated mice surviving over 60 days (median survival=60 days). Comparing TrQβ@b-3WJsiEGFR+Let-7g and 2Gy X ray irradiation treatment group with other treatment groups, significant differences (p<0.05) were observed in tumor volume change on Day 41 and in survival using Student's t test and log-rank test (Table 51).

TABLE S1
Log-rank analysis result of survival study.

| | Chi-Square | Prob > Chi- | |
|---|---|---|---|
| Groups | (log-rank) | Square | p value |
| Control v.s. | 0.562 | 0.453 | 0.453 |
| TrQβVLPs | | | (No significant |
| | | | difference) |
| Control v.s. | 0.035 | 0.850 | 0.852 |
| 2Gy X-ray | | | (No significant |
| | | | difference) |
| Control v.s. | 4.305 | 0.038 | 0.038 |
| Carblplatin + | | | (Significant |
| 2Gy X-ray | | | difference) |
| Control v.s. | 12.035 | 0.0005 | 0.0005 |
| rQβ@b-3WJsiEGFR + | | | (Significant |
| Let-7g and 2Gy X-ray | | | difference) |
| Control v.s. | 12.035 | 0.0005 | 0.0005 |
| TrQβ@b-3WJsiEGFR + | | | (Significant |
| Let-7g and 2Gy X-ray | | | difference) |
| 2Gy X-ray v.s. | 17.254 | 0.0002 | 0.00003 |
| TrQβ@b-3WJsiEGFR + | | | (Significant |
| Let-7g and 2Gy X-ray | | | difference) |
| Carblplatin + 2Gy X-ray | 1.970 | 0.160 | 0.160 |
| v.s. | | | (No significant |
| rQβ@b-3WJsiEGFR + | | | difference) |
| Let-7g and 2Gy X-ray | | | |
| Carblplatin + 2Gy X-ray | 4.502 | 0.034 | 0.033 |
| v.s. | | | (Significant |
| TrQβ@b-3WJsiEGFR + | | | difference) |
| Let-7g and 2Gy X-ray | | | |
| rQβ@b-3WJsiEGFR + | 14.640 | 0.0001 | 0.0001 |
| Let-7g and 2Gy X-ray | | | (Significant |
| v.s. | | | difference) |
| TrQβ@b-3WJsiEGFR + | | | |
| Let-7g and 2Gy X-ray | | | |

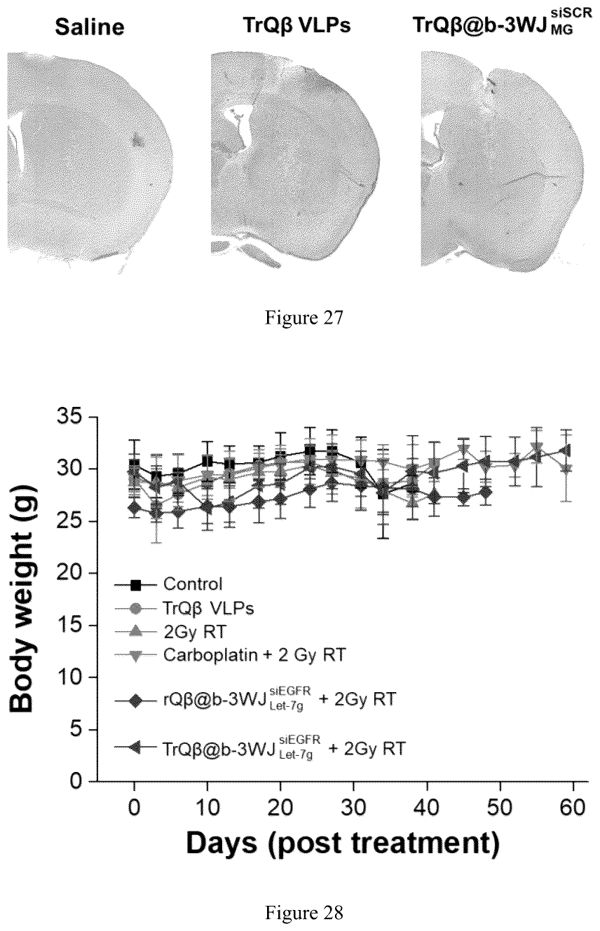

The increase in the median survival time (IST median; in %) of the X-ray irradiation group was set as the standard baseline (IST median=100%); the IST median for the mice that received the combination of rQβ@b-3WJsiEGFR+Let-7g pretreatment with 2Gy of Xray irradiation increased to 132%, indicating that the rQβ@b-3WJsiEGFR+Let-7g can inhibit DNA repair process to enhance the efficiency of radiotherapy. However, it is worth noting that the IST median increased nearly 1.5-fold to 194% when the mice received the combination of rQβ@b-3WJsiEGFR+Let-7g and TrQβ@b-3WJsiEGFR+Let-7g pretreatment with 2Gy of X-ray irradiation, which means that the TrQβ@b-3WJsiEGFR+Let-7g can enter tumor tissue cells more efficiently compared with rQβ@b-3WJsiEGFR+Let-7g and the therapeutic efficiency of radiotherapy was substantially promoted by TrQβ@b-3WJsiEGFR+Let-7g-based gene-silencing-enhanced radiotherapy. Finally, the toxicity of QβVLP-based therapeutics (TrQβ VLPs and TrQβ@b-3WJsiSCR+MG) was evaluated to ensure their safety. Histopathologic examination revealed no obvious differences in the brain tissue sections of the saline, TrQβ VLPs, and TrQβ@b-3WJsiSCR+MG-treated mice, indicating an absence of neurological toxicity during CED infusion of Qβ VLP-based therapeutics over a 5-day observation period (FIG. 27). The previous blood biochemical analyses showed that the Qβ VLP-based therapeutics would not cause both liver and renal functions, and there were no signs of inflammation or antigenicity. Additionally, no significant body weight loss was observed in the mice treated with TrQβ VLPs or rQβ@b-3WJsiEGFR+Let-7g or TrQβ@b-3WJsiEGFR+Let-7g (FIG. 28). The overall results indicate that TrQβ@b-3WJsiEGFR+Let-7g based genetic therapeutics are efficient and safe enough for use with low-dose X-ray irradiation in future clinical brain tumor treatment modalities.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

1. A bioengineered bacteriophage-like nanoparticle, rQβ@b-3WJ, comprising a Qβ capsid and a 3WJ RNA scaffold (b-3WJ), in which the b-3WJ is a three-way junction motif integrated with (1) a Qβ phage capsid binding hairpin; (2) a light-up aptamer; and (3) a first structural siRNA element (siRNA₁); wherein the Qβ phage capsid binding hairpin is further integrated with a second structural siRNA element (siRNA₂).

2. The bioengineered bacteriophage-like nanoparticle of claim 1, which further comprises a transactivating transcriptional activator (TAT) peptide, which is conjugated with the Qβ capsid.

3. The bioengineered bacteriophage-like nanoparticle of claim 1, wherein the light-up aptamer is malachite green (MG) aptamer or broccoli aptamer.

4. The bioengineered bacteriophage-like nanoparticle of claim 1, wherein the structural siRNA element comprises EGFR siRNA, LUC2 siRNA, and Let-7g.

5. The bioengineered bacteriophage-like nanoparticle of claim 1, wherein the 3WJ RNA scaffold has an RNA sequence selected from the group consisting of SEQ ID NO:2 and SEQ ID NO:3.

6. A pharmaceutical composition, comprising a therapeutically effective amount of the bioengineered bacteriophage-like nanoparticle, rQβ@b-3WJ, set forth in claim 1, in which the first siRNA element is a nucleic acid drug.

7. The pharmaceutical composition of claim 6, comprising a therapeutically effective amount of the bioengineered bacteriophage-like nanoparticle, rQβ@b-3WJ, in which the second siRNA element is a nucleic acid drug.

8. The pharmaceutical composition of claim 6, wherein the 3WJ RNA scaffold has an RNA sequence selected from the group consisting of SEQ ID NO:2 and SEQ ID NO:3.

9. A pharmaceutical composition for treating glioblastomas, comprising a therapeutically effective amount of the bioengineered bacteriophage-like nanoparticle, rQβ@b-3WJ, set forth in claim 1, in which the first structure siRNA element is EGFR siRNA and the second structure siRNA element is miRNA Let-7g.

10. The pharmaceutical composition of claim 9, wherein the 3WJ RNA scaffold has an RNA sequence selected from the group consisting of SEQ ID NO:2 and SEQ ID NO:3.
